(19) United States
(12) Patent Application Publication
(10) Pub. No.: US 2026/0069745 A1
(43) Pub. Date: Mar. 12, 2026

(54) BIOLOGIC COMPOSITION FOR PROMOTING WOUND HEALING AND A METHOD OF PREPARING THE SAME

(71) Applicant: Center for Neuromusculoskeletal Restorative Medicine Limited, Hong Kong (CN)

(72) Inventors: Anna Maria Blocki, Hong Kong (CN); Kwok Keung Lit, Hong Kong (CN)

(21) Appl. No.: 19/298,252

(22) Filed: Aug. 13, 2025

Related U.S. Application Data
(60) Provisional application No. 63/693,791, filed on Sep. 12, 2024.

(51) Int. Cl. A61L 27/36; A61L 27/20; A61L 27/54; C12N 5/0775

(52) U.S. Cl. CPC A61L 27/3633; A61L 27/20; A61L 27/3691; A61L 27/54; C12N 5/0663; A61L 2300/412; A61L 2300/414; A61L 2300/426; A61L 2430/34; C12N 2500/02; C12N 2500/98

(57) ABSTRACT

A biologic composition for promoting wound healing includes: a human mesenchymal stem cell (MSC)-derived extracellular matrix (ECM) biologic component, and a negatively charged polymer, combined with the biologic component to form the biologic composition. A method of preparing the biologic composition thereof includes: (i) isolating human mesenchymal stem cells (MSC) and subjecting the MSC to hypoxia-priming in a xeno- and/or serum-free, or chemically defined medium to stimulate the production of an MSC-derived biologic component with pro-angiogenic factors; (ii) aggregating and co-precipitating the MSC-derived biologic component with a negatively charged polymer into a pericellular space of the MSC to produce an aggregated biologic component; (iii) assembling the aggregated biologic component and the negatively charged polymer into an insoluble extracellular matrix (ECM) by the MSC; and (iv) decellularizing the insoluble ECM to obtain the biologic composition comprising MicroParticles of Solidified Secretome (MIPSOS).

### TECHNICAL FIELD

This invention relates to a biologic composition for promoting wound healing, and a method of preparing the same. In particular, the present invention is related to a composition for promoting wound healing comprising a human mesenchymal stem cell (MSC)-derived extracellular matrix (ECM) biologic component, and a negatively charged polymer, and wherein the biologic composition improves pro-angiogenic bioactivity.

### BACKGROUND

Wound healing is a biological process that repairs and restores damaged tissue after injury. Wound healing is affected by a number of factors, including the nature and severity of the wound, the affected individual's overall health and underlying health conditions, and any specificities of the injury. Chronic wounds have a profound impact on the quality of life and affect a large number of people. The management of wounds has a significant impact on global healthcare, with the continued burden of chronic conditions such as diabetes and obesity on the rise worldwide. Risk factors such as vascular issues, hypertension, chronic kidney disease and diabetes can have a profound impact on wound healing and can put significant pressure on healthcare systems due to prolonged treatment times, risk of infection, complexity, and associated costs. The treatment time and severity can vary and chronic wounds, such as diabetic ulcers, require extensive management and ongoing care, often involving advanced therapies, specialist clinicians, and specialized equipment.

Chronic wounds typically persist for longer amounts of time, for example for more than three months or more, and are injuries that are characterized as failing to heal normally. They can often result from chronic conditions like diabetes, pressure ulcers, or venous insufficiency. Typically, the wound healing is reduced or impeded by underlying factors in a patient, including circulation, infection, or other health issues. Chronic wounds frequently require long-term, frequent, and specialised care and is often expensive, time-consuming, and can have a considerable impact on mental health and quality of life. They also sometimes require surgical intervention, or at least repeated dressings, medication, and medical attention.

The treatment of wounds continues to be a clinical, social and economic challenge. A biologic composition that promotes wound healing is desired.

### SUMMARY OF THE INVENTION

Wound healing is a process through which damaged tissue is restored and repaired. Effective wound healing requires a coordinated response involving proliferation, cell migration, and tissue remodelling. Effective wound healing is critical in conditions such as, for example, traumatic injuries, chronic infections, diabetes, and surgical wounds, to prevent complications and promote recovery.

The claimed invention provides a biologic composition comprising human mesenchymal stem cell (MSC)-derived ECM. The biologic composition advantageously is free from xenogeneic and serum components, and incorporates a secretome enriched with pro-angiogenic factors and a negatively charged polymer. The biologic composition has enhanced pro-angiogenic activity and accelerates wound healing with improved cellular infiltration, granulation tissue formation, and nerve growth, thus providing a promising therapy for wound healing. The biologic composition beneficially demonstrates superior therapeutic efficacy compared to tissue-derived ECM products, with improvements in wound closure rates and enhanced pro-angiogenic bioactivity.

According to a first aspect of the invention, there is provided a composition for promoting wound healing, comprising:
a human mesenchymal stem cell (MSC)-derived extracellular matrix (ECM) biologic component, and
a negatively charged polymer,
wherein the negatively charged polymer is combined with the biologic component to form the biologic composition.

In a preferred example embodiment, the biologic composition is an extracellular matrix (ECM)-negatively charged polymer composite biologic. In a most preferred embodiment, the biologic composition is MicroParticles of Solidified Secretome (MIPSOS). The inventors advantageously developed a biologic composition with augmented pro-angiogenic potential based on human mesenchymal stem cell (MSC)-derived ECM for the treatment of wounds, for example diabetic wound healing, chronic wound healing, and the treatment of ischemic tissues. In particular, the biologic composition promotes healing of normal wounds, non-healing chronic wounds, chronic wounds, diabetic wounds, or a combination thereof. For example, skin wounds or other kinds of wounds. In another embodiment, the MIPSOS is synthesised under hypoxic conditions in a xeno-free, serum-free, and chemically defined medium to provide enhanced pro-angiogenic bioactivity. The inventors advantageously developed a biologic composition with augmented pro-angiogenic potential based on human mesenchymal stem cell (MSC)-derived ECM for the treatment of wounds, for example diabetic wound healing, chronic wound healing, and the treatment of ischemic tissues. In particular, the biologic composition promotes healing of normal wounds, non-healing chronic wounds, chronic wounds, diabetic wounds, or a combination thereof. For example, the wounds include skin wounds or other kinds of wounds.

In a further example embodiment, the biologic composition is free of xeno- and serum-derived components.

For example, the negatively charged polymer is a sulphated polymer. In a further example, the negatively charged polymer is a polyglucose polymer. In a preferred example, the negatively charged polymer is dextran sulfate (DxS). In an example embodiment, the negatively charged polymer is present at a concentration of 10 μg/ml during ECM synthesis.

In an example embodiment, the negatively charged polymer facilitates the deposition of an ECM with pro-angiogenic factors. For example, the biologic composition is produced by culturing MSCs in the presence of the negatively charged polymer and ascorbate whereby the negatively charged polymer aggregates and co-precipitates ECM proteins into the pericellular space and facilitates ECM assembly. In one embodiment, ascorbate is present at a concentration of 30 μg/ml during ECM synthesis. DxS, for example, is a non-toxic polyglucose polymer, and ECM co-assembly with DxS leads to an accumulation of bioactive molecules in the biologic composition. Further, the negatively charged polymer advantageously binds bioactive factors. For example, the DxS is a heparan sulfate mimetic that binds bioactive factors. In a preferred embodiment, the negatively charged polymer is 500 kDa dextran sulfate sodium salt from Leuconostoc spp which promotes the deposition and retention of bioactive factors within the ECM for augmented bioactivity. For example, one or more bioactive factors are selected from growth factors, cytokines, and chemokines. For example, the bioactive factors include enhanced levels of vascular endothelial growth factor A (VEGF-A).

In an example embodiment, the biologic component is a secretome with pro-angiogenic factors. Most preferably, the biologic component is a secretome whereby the secretome is aggregated and co-precipitated with the negatively charged polymer, for example DxS, into pericellular space of hypoxia-primed MSC to form an insoluble ECM that is decellularised and processed into the biologic composition.

The biologic composition is effective at wound healing and the composition and/or structure of the ECM may be tailored as desired, for example to provide a broader array of pro-angiogenic factors in increased quantities. The biologic component is derived from cell culture, whereby the cells are MSCs. In an example embodiment, the MSCs from which the biologic component is derived are cultured in a xeno-free, and/or serum-free, or chemically defined medium. In yet another example embodiment, the MSCs are cultured in R: Stem medium, which is a xeno-free, serum-free, and chemically defined medium that provides enhanced pro-angiogenic bioactivity. In a further example embodiment, the MSCs from which the biologic component is derived are cultured in a xeno-free, and/or serum-free, or chemically defined medium under hypoxic conditions. In one embodiment, the hypoxic conditions comprise 5% O₂ tension maintained for a period of 6 days without media changes.

In one example, the MSCs are cultured under hypoxic conditions in R: Stem medium, which synergistically enhances the pro-angiogenic properties of the resulting biologic composition, including increased VEGF-A content and superior therapeutic efficacy in wound healing applications.

In an example embodiment, the biologic composition is obtained by one or more of: mechanical collection, solubilization, and lyophilization. Mechanical collection includes but is not limited to, for example, centrifugation or filtration. Solubilization, includes dissolving the biologic composition into a liquid form for gel or injectable applications. Lyophilization allows for the removal of water from the biologic composition by freeze-drying to prepare a stable powder form. For example, the biologic composition is processed by mechanical scraping, resuspension in deionized water, and lyophilization into fine powder form for storage and reconstitution.

In an example embodiment, the biologic composition is stored in a stabilized form and retains bioactivity for several weeks to months when stored frozen and desiccated, providing a stable and effective therapeutic product.

In a further example embodiment, the biologic composition further comprises a carrier component to facilitate application and retention of the biologic composition at a target site. For example, the carrier component is selected from hydrogels, sponges, sheets, powders, wound cleansers, antiseptic solutions, saline solution, topical antibiotics, barrier creams, emollients, hydrocolloid creams or dressings, foam dressings, antimicrobial dressings collagen dressings, dressings with a foam and film layer, or combinations thereof. In a preferred embodiment, the carrier component is a fibrinogen solution that is polymerised with thrombin in situ to form a stable fibrin matrix for wound delivery.

In an example embodiment, the biologic composition is delivered at a concentration of 10 mg/ml in the carrier component for topical application to wound sites.

The biologic composition of the claimed invention advantageously promotes angiogenesis in vitro and in vivo and accelerates re-epithelization and wound closure. The biologic composition of the claimed invention demonstrates superior wound healing efficacy with wound closure rates of up to 85% at 17 days in diabetic wound healing models. The promotion of angiogenesis results in enhanced bloody supply as new blood vessels increase the supply of oxygen and nutrients to the wound site that are vital for tissue repair and restoration. Additional advantages of enhanced angiogenesis include increased migration of fibroblasts and epithelial cells to the wound site, thus supporting the production of ECM and collagen by supply of necessary bioactive factors. Angiogenesis also reduces the infection risk and helps in the delivery of immune cells to the wound site. The acceleration of re-epithelialization reduced inflammation at the wound site by reducing the exposure of the underlying tissues to pathogens. The formation of a new epithelial layer also protects the wound and improves resistance to mechanical stress at the wound site, whilst also accelerating the overall healing process.

In one embodiment, the biologic composition promotes one or more of the following in skin wounds: wound closure, revascularisation, cellular infiltration, ECM deposition, granulation tissue formation, and re-innervation. For example, in diabetic skin wounds. In particular, the biologic composition promotes enhanced cellular infiltration, increased vessel density as measured by CD31 staining, and improved re-epithelialization as demonstrated by cytokeratin-1 expression.

According to a second aspect of the invention, there is provided a method for treating diabetic wounds, comprising administering to a wound an effective amount of the biologic composition described above.

In an example embodiment, the biologic composition is applied topically to a wound site. In a further example embodiment, the administration of the biologic composition accelerates wound re-epithelization and closure. In a preferred embodiment, the biologic composition accelerates wound closure by up to 40%.

According to a third aspect of the invention, there is provided a method of preparing a biologic composition for promoting wound healing, comprising the steps of:
(i) isolating human mesenchymal stem cells (MSC) and subjecting the MSC to hypoxia-priming in a medium to stimulate the production of an MSC-derived biologic component with pro-angiogenic factors;
(ii) aggregating and co-precipitating the MSC-derived biologic component with a negatively charged polymer into a pericellular space of the MSC to produce an aggregated biologic component;
(iii) assembling the aggregated biologic component and the negatively charged polymer into an insoluble extracellular matrix (ECM) by the MSC; and
(iv) decellularizing the insoluble ECM to obtain the biologic composition comprising MicroParticles of Solidified Secretome (MIPSOS).

In one embodiment, the method further comprises the step of culturing the MSCs for a period of at least 5 days without media changes under the hypoxic conditions.

In an example embodiment, the negatively charged polymer is a sulphated polymer. In a further example, the negatively charged polymer is a polyglucose polymer. Preferably, the negatively charged polymer is dextran sulfate (DxS). For example, the DxS is present at a concentration of 10 μg/ml during ECM synthesis.

In a preferred example embodiment, the medium is a xeno-free, and/or serum-free, or chemically defined medium. In one embodiment, the medium is R: Stem medium, which is xeno-free, serum-free, and chemically defined, and provides enhanced pro-angiogenic bioactivity.

In one example, the hypoxic conditions comprise 5% oxygen tension maintained throughout the culture period.

In yet another example embodiment, the medium further comprises ascorbic acid at a concentration of 30 μg/ml to facilitate ECM deposition and assembly.

In another example of the embodiment, the method further comprises processing the insoluble ECM, by chemical or mechanical means, to obtain the biologic composition comprising MIPSOS. In one embodiment, the processing comprises decellularization using sodium deoxycholate treatment followed by DNase I treatment to remove residual DNA, followed by mechanical scraping, resuspension in deionized water, and lyophilization to obtain fine powder microparticles.

In an example embodiment, the decellularization comprises incubating the culture at room temperature for 15 minutes with 0.15% (w/V) sodium deoxycholate supplemented with 0.175% (V/V) protease inhibitor cocktail in deionized water, followed by incubation at 37° C. for 15 minutes with 0.04 mg/ml DNase I in phosphate buffered saline.

For example, the method further comprises the step of incorporating a carrier means to facilitate application of the biologic composition comprising MIPSOS at a wound site.

For example, the carrier means is selected from groups consisting of hydrogels, sponges, sheets, powders, wound cleansers, antiseptic solutions, saline solution, topical antibiotics, barrier creams, emollients, hydrocolloid creams or dressings, foam dressings, antimicrobial dressings collagen dressings, dressings with a foam and film layer, or combinations thereof. For example, the carrier means is a fibrinogen solution at a final concentration of 10 mg/ml in 0.9% NaCl, which is polymerized with thrombin at a final concentration of 5 U/ml to form a stable fibrin matrix for wound delivery.

Preferably, the wherein the negatively charged polymer is 500 kDa dextran sulfate sodium salt from Leuconostoc spp.

In an embodiment, the biologic composition comprising MIPSOS is stored in a stabilized form and can be reconstituted or applied directly to the wound site as needed. The biologic composition retains bioactivity for several weeks to months when stored frozen and desiccated, providing a desirable therapeutic product with stable performance characteristics.

The claimed method beneficially provides a biologic composition that is configured to enhance wound healing in diabetic wounds by promoting angiogenesis, cellular infiltration, and granulation tissue formation. The method advantageously provides a biologic composition with superior therapeutic efficacy with up to 40% improvement in wound closure rates.

The claimed biologic composition and method of preparation of the same advantageously provides a wound healing therapy that is free of xeno- and serum-derived components and promotes angiogenesis and accelerates healing whilst minimising the risk of infection.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present invention will now be described, by way of example, with reference to the accompanying drawings in which:

FIG. 1 is a flow chart illustrating a method of preparing a biologic composition for promoting wound healing in accordance with an example embodiment.

FIG. 2 is a schematic illustrating a method of preparing a biologic composition for promoting wound healing in accordance with an example embodiment.

FIG. 3A is a graph showing the quantification of mesenchymal stem cell (MSC) proliferation in various xeno-free and serum-free media.

FIG. 3B shows immunocytochemistry images for fibronectin and collagen I (major ECM structural components) deposited by MSCs when cultured in various cell culture media.

FIG. 3C is a graph showing the quantification of area coverage of stained fibronectin.

FIG. 3D is a graph showing the quantification of area coverage of stained collagen I.

FIG. 3E is a graph showing human umbilical vein endothelial cell (HUVEC) proliferation on decellularized MSC-derived ECM-based substrates, synthesised in various media.

FIG. 4A shows immunocytochemistry staining for transcription factor hypoxia-inducible factor 1-alpha (HIF-1-α) in MSCs cultured in DMEM/FBS or R: Stem under hypoxic or normoxic conditions for up to 6 days (nuclei were stained with DAPI).

FIG. 4B is a graph illustrating an assessment of cell proliferation MSCs by CCK-8 assay after 6 days of culture.

FIG. 4C shows live/dead cell staining of MSCs after 6 days of culture.

FIG. 5A shows immunocytochemistry staining for fibronectin in MSCs cultured in DMEM/FBS or R: Stem under hypoxic or normoxic conditions for 6 days.

FIG. 5B is a graph showing stained area coverage for fibronectin in MSCs cultured in DMEM/FBS or R: Stem under hypoxic or normoxic conditions for 6 days.

FIG. 5C shows immunocytochemistry staining for collagen type I in MSCs cultured in DMEM/FBS or R: Stem under hypoxic or normoxic conditions for 6 days.

FIG. 5D is a graph showing stained area coverage for collagen type I in MSCs cultured in DMEM/FBS or R: Stem under hypoxic or normoxic conditions for 6 days.

FIG. 5E shows immunocytochemistry staining for VEGF-A in MSCs cultured in DMEM/FBS or R: Stem under hypoxic or normoxic conditions for 6 days. Nuclei were stained with DAPI.

FIG. 5F is a graph showing stained area coverage for VEGF-A in MSCs cultured in DMEM/FBS or R: Stem under hypoxic or normoxic conditions for 6 days.

FIG. 5G is a gradient SDS-PAGE analysis of decellularized matrices derived from MSCs cultured in DMEM/FBS or R: Stem under hypoxic or normoxic conditions for 6 days. Red solid frame highlights protein bands prominent for matrices deposited under hypoxic conditions and the red dashed boxes highlight protein bands prominent in matrices derived from MSCs cultured in DMEM/FBS.

FIG. 5H shows immunocytochemistry staining and quantification of stained area coverage for VEGF-A.

FIG. 6A is a graph showing the proliferation of HUVECs seeded on decellularized matrices assembled in DMEM/FBS or R: Stem under normoxic or hypoxic conditions evaluated by a CCK-8 assay after 3 days.

FIG. 6B is a graph showing cumulative HUVEC spheroid sprout length in which HUVEC spheroids were overlaid in collagen I hydrogels over matrices assembled in DMEM/FBS or R: Stem under normoxic or hypoxic conditions and allowed to sprout for 2 days.

Samples were stained with phalloidin, imaged and cumulative sprouting length was measured.

FIG. 7A is a schematic showing the biologic composition (MIPSOS) preparation and application into full-thickness skin tail wounds of db/db mice.

FIG. 7B shows representative images of wounds over a time course of 17 days. The yellow lines indicate a wound outline.

FIG. 7C is a graph showing measured tail skin wound areas plotted over time.

FIG. 7D is a graph showing average healing rate quantified from wound areas over time.

FIG. 7E is a graph showing measured wound areas plotted over time.

FIG. 7F shows representative images of wounds over a time course of 17 days.

FIG. 8A shows immunofluorescence staining of day 17 wounds for cytokeratin 1 (K1), a marker for mature epidermis, and DAPI.

FIG. 8B1 is a semi-quantitative assessment of K1 area coverage for fibrin hydrogel only, GraftJacket®, Normoxic DMEM/FBS MIPSOS, Hypoxic DMEM/FBS MIPSOS, and Hypoxic R: Stem MIPSOS.

FIG. 8B2 is a semi-quantitative assessment of the remaining epithelial gap on day 17 for fibrin hydrogel only, GraftJacket®, Normoxic DMEM/FBS MIPSOS, Hypoxic DMEM/FBS MIPSOS, and Hypoxic R: Stem MIPSOS.

FIG. 8C shows H&E staining and Masson Trichrome staining of granulation tissue of day 17 wounds.

FIG. 8D1 is a representative H&E staining of whole tail cross-section.

FIG. 8D2 is a semi-quantitative analysis of hematoxylin stained nuclei in H&E stained granulation tissues.

FIG. 8D3 shows H&E staining of tail sections harbouring wounds. Granulation tissue is outline by black dashed lines and selected areas (red dashed squares) are enlarged on the right side.

FIG. 8E1 is a representative Masson's Trichrome staining of a whole tail cross-section.

FIG. 8E2 is a graph illustrating a semi-quantitative analysis of blue-stained collagen fibers in Masson's Trichrome stained granulation tissues.

FIG. 8E3 shows images of Masson's Trichrome staining of tail sections harbouring wounds.

FIG. 9A shows representative images of CD31, iNOS/CD11b and CD206/CD11b stained granulation tissue of wounds treated with fibrin hydrogel, GraftJacket®, Normoxic DMEM/FBS MIPSOS, Hypoxic DMEM/FBS MIPSOS, and Hypoxic R: Stem MIPSOS.

FIG. 9B1 is a graph showing the quantification of area stained positively for CD31.

FIG. 9B2 is a graph showing the quantification of area stained positively for CD31 per field of view (FOV).

FIG. 9B3 shows representative images of CD31 and DAPI staining of a day 17 wound.

The interface between wound and scab area is indicated by a white dashed line.

FIG. 9B4 shows representative images of CD31 and DAPI staining of a day 17 wound.

FIG. 9B5 shows representative images of F4/80, iNOS and CD206 stained wounds on day 17. Interfaces between wound and scab areas are indicated by white dashed lines.

FIG. 9C1 is a graph showing the quantification of cell stained double positive for iNOS/CD11b per field of view (FOV).

FIG. 9C2 is a graph showing the quantification of cell stained for iNOS per FOV.

FIG. 9D1 is a graph showing the quantification of cell stained double positive for CD206/Cd11b per field of view (FOV).

FIG. 9D2 is a graph showing the quantification of cell stained for CD206 per FOV.

FIG. 9D3 is a graph showing the quantification of cell stained for F4/80 per FOV.

FIG. 10A shows representative images of immunohistochemical staining for β-tubulin III (TubB3) of wound tissues treated with fibrin hydrogel, GraftJacket®, Normoxic DMEM/FBS MIPSOS, Hypoxic DMEM/FBS MIPSOS, and Hypoxic R: Stem MIPSOS.

FIG. 10B is a graph showing the quantification of integrated density of TubB3 per fold change.

FIG. 11A is a graph showing mesenchymal stem cells (MSC) metabolic activity when cultured in DMEM/FBS, CnT, NutriStem, PromoCell, StemXVivo, R: Stem media.

FIG. 11B is a graph showing MSC metabolic activity following culture in DMEM/FBS, CnT, NutriStem, PromoCell, StemXVivo, R: Stem media.

FIG. 11C is a graph showing MSC metabolic activity following culture in DMEM/FBS, CnT, NutriStem, PromoCell, StemXVivo, R: Stem media.

FIG. 11D is a graph showing MSC metabolic activity following culture in DMEM/FBS, CnT, NutriStem, PromoCell, StemXVivo, R: Stem media.

FIG. 11E is a graph showing average MSC metabolic activity following culture in DMEM/FBS, CnT, NutriStem, PromoCell, StemXVivo, R: Stem media.

FIG. 12A is a graph showing human umbilical vein endothelial cell (HUVEC) metabolic activity following culture in TCP, DMEM/FBS, CnT, NutriStem, PromoCell, StemXVivo, R: Stem media.

FIG. 12B is a graph showing HUVEC metabolic activity following culture in TCP, DMEM/FBS, CnT, NutriStem, PromoCell, StemXVivo, R: Stem media.

FIG. 12C is a graph showing HUVEC metabolic activity following culture in TCP, DMEM/FBS, CnT, NutriStem, PromoCell, StemXVivo, R: Stem media.

FIG. 12D is a graph showing HUVEC metabolic activity following culture in TCP, DMEM/FBS, CnT, NutriStem, PromoCell, StemXVivo, R: Stem media.

FIG. 12E is a graph showing HUVEC metabolic activity following culture in TCP, DMEM/FBS, CnT, NutriStem, PromoCell, StemXVivo, R: Stem media.

FIG. 12F is a graph showing HUVEC metabolic activity following culture in TCP, DMEM/FBS, CnT, NutriStem, PromoCell, StemXVivo, R: Stem media.

FIG. 12G is a graph showing average HUVEC metabolic activity following culture in TCP, DMEM/FBS, CnT, NutriStem, PromoCell, StemXVivo, R: Stem media.

FIG. 13A shows images of MSC-derived ECM-based substrates deposited in various media before decellularization.

FIG. 13B shows images of MSC-derived ECM-based substrates deposited in various media after decellularization.

FIG. 13C shows images of MSC-derived ECM-based substrates deposited in DMEM/FBS or R: Stem medium under hypoxic or normoxic cell culture conditions before decellularization.

FIG. 13D shows images of MSC-derived ECM-based substrates deposited in DMEM/FBS or R: Stem medium under hypoxic or normoxic cell culture conditions after decellularization.

FIG. 14A is a graph showing MSC metabolic activity following culture in DMEM/FBS and R: Stem media under hypoxic or normoxic cell culture conditions.

FIG. 14B is a graph showing MSC metabolic activity following culture in DMEM/FBS and R: Stem media under hypoxic or normoxic cell culture conditions.

FIG. 14C is a graph showing MSC metabolic activity following culture in DMEM/FBS and R: Stem media under hypoxic or normoxic cell culture conditions.

FIG. 14D is a graph showing average MSC metabolic activity following culture in DMEM/FBS and R: Stem media under hypoxic or normoxic cell culture conditions.

FIG. 15A is a graph showing HUVEC metabolic activity following culture in TCP, and DMEM/FBS and R: Stem media under hypoxic or normoxic cell culture conditions.

FIG. 15B is a graph showing HUVEC metabolic activity following culture in TCP, and DMEM/FBS and R: Stem media under hypoxic or normoxic cell culture conditions.

FIG. 15C is a graph showing HUVEC metabolic activity following culture in TCP, and DMEM/FBS and R: Stem media under hypoxic or normoxic cell culture conditions.

FIG. 15D is a graph showing HUVEC metabolic activity following culture in TCP, and DMEM/FBS and R: Stem media under hypoxic or normoxic cell culture conditions.

FIG. 15E is a graph showing HUVEC metabolic activity following culture in TCP, and

DMEM/FBS and R: Stem media under hypoxic or normoxic cell culture conditions.

FIG. 15F is a graph showing HUVEC metabolic activity following culture in TCP, and DMEM/FBS and R: Stem media under hypoxic or normoxic cell culture conditions.

FIG. 15G is a graph showing average HUVEC metabolic activity following culture in TCP, and DMEM/FBS and R: Stem media under hypoxic or normoxic cell culture conditions.

FIG. 16A shows representative images of iNOS, CD11b stained granulation tissue treated with GraftJacket®, normoxic DMEM MIPSOS, hypoxic DMEM MIPSOS, and hypoxic R: Stem MIPSOS.

FIG. 16B is a graph showing CD206, CD11b stained granulation tissue treated with the different materials.

### DETAILED DESCRIPTION OF THE PREFERRED EMBODIMENT

Chronic wounds, particularly chronic diabetic wounds, often suffer from complexities such as impaired re-vascularization and this can lead to issues such as impaired tissue repair, prolonged inflammation, and nutrient deprivation. Due to the resulting poor tissue perfusion, ischemic tissues are continuously injured by low oxygen tension and nutrient deprivation, leading to prolonged inflammation, increased oxidative stress, dysregulated extracellular matrix (ECM) synthesis, decreased cell proliferation and impaired re-epithelization. Re-establishing sufficient blood supply to the affected tissues is thus an important step in addressing issues in wound healing.

Other issues include, but are not limited to, ischaemia which prolongs inflammation and delays repair, chronic inflammation, infection, decreased cell proliferation and migration which slows wound closure and regeneration, dysregulated ECM synthesis which results in poor tissue integrity and function, and neuropathy which can result in an increased risk of chronic wounds and infection.

Various therapeutic approaches have been developed in the treatment of wounds, but they suffer from limited clinical efficacy and are associated with risks and side effects. Growth factor therapies, aim to supply exogenous pro-angiogenic factors to promote re-vascularization. Yet, these soluble factors have short half-lives and high diffusion rates, thus are required to be supplied in high doses, resulting in risks of carcinogenesis and off-site effects. Further, the provision of a single or few factors lacks the necessary biocomplexity to effectively orchestrate and regulate angiogenesis. Cellular therapies provide a more holistic approach, where MSCs promote angiogenesis and regeneration through a wide range of secreted factors. However, the inflamed and oxygen-deprived host environment hinders the survival and engraftment of MSCs, while the low rate of cell retention limits the clinical efficacy of MSC therapies. Tissue-derived decellularized ECM-based biomaterials, such as GraftJacket®, have been utilised as wound healing therapeutics. However, such materials are derived from decellularized human cadaveric dermis and face limitations such as limited availability, risk of immunogenicity, batch-to-batch variability and the inability to tailor bioactivity for desired properties. Other biomaterials that rely on the use of cadaveric sources and incomplete removal of immunogenic components carry the inherent of risk of disease transmission and undesirable immune responses.

Thus, current therapies face limitations like short half-lives where rapid diffusion requires high doses, high infection rates, and poor cell survival.

The inventors have developed a biologic composition comprising MicroParticles of Solidified Secretome (MIPSOS). The biologic composition is derived from mesenchymal stem cell (MSC)-derived extracellular matrix (ECM). The biologic composition utilises a negatively charged polymer to aggregate ECM proteins and enhance their pro-angiogenic bioactivity. The biologic composition of the claimed invention is processed into fine particles with enhanced pro-angiogenic properties, thus accelerating wound healing in vitro and in vivo effectively. The biologic composition is also advantageously synthesised in xeno- and serum-free conditions, thereby allowing for enhanced reproducibility and reduced immunogenic risk. The biologic composition of the claimed invention demonstrates superior therapeutic efficacy compared to clinically approved tissue-derived ECM products, with quantified improvements in wound closure rates and enhanced angiogenic bioactivity. The biologic composition of the claimed invention provides an effective therapeutic with high potential and application in wound healing.

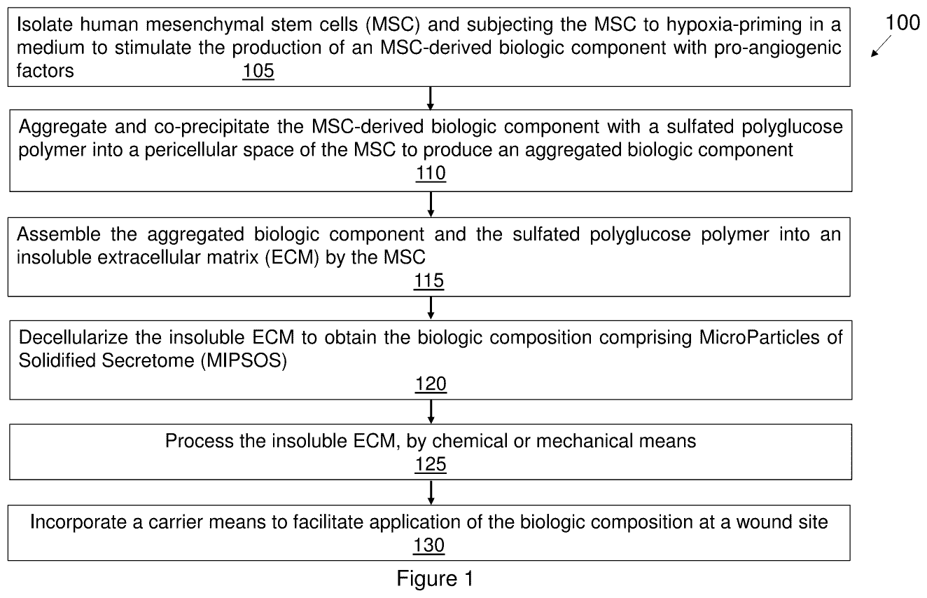

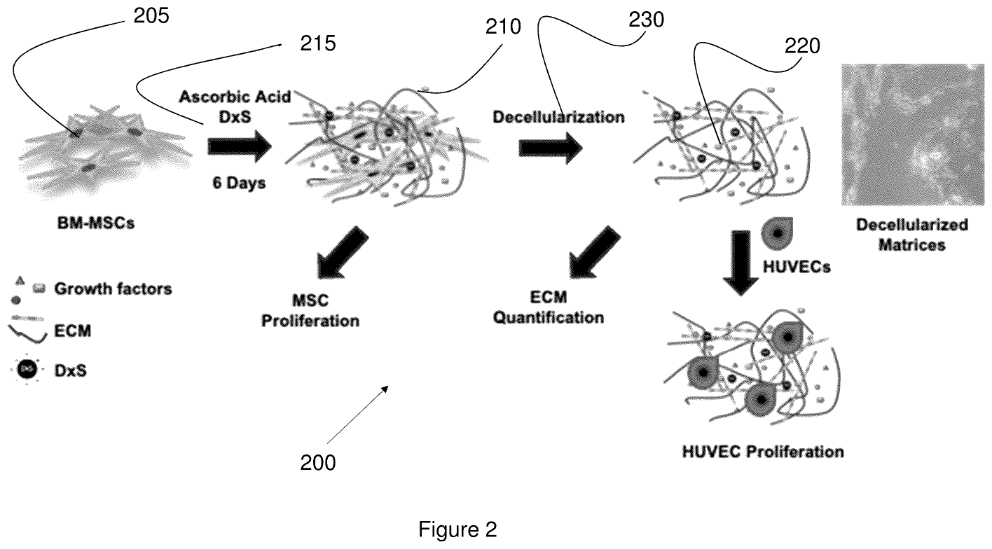

With reference to FIG. 1 100 and FIG. 2 200, embodiments of the present invention are illustrated providing a biologic composition 220 for wound healing and a method 200 of preparing the same.

With reference to FIG. 1 100 and FIG. 2 200, an embodiment of the present invention is arranged to provide a biologic composition 220 for promoting wound healing and a method 100 of preparing the same. The biologic composition 220 includes a human mesenchymal stem cell (MSC)-derived extracellular matrix (ECM) biologic component 210, and a negatively charged polymer 215, wherein the negatively charged polymer 215 is combined with the biologic component 210 to form the biologic composition 220. In an example embodiment, the negatively charged polymer is a sulphated polymer. In another example embodiment, the negatively charged polymer is a polyglucose polymer. In a preferred embodiment, the negatively charged polymer is dextran sulfate (DxS).

The mesenchymal stem cells (MSCs) 205 from which the biologic component 210 is derived are first isolated and subjected to hypoxia priming in a xeno-free, and/or serum-free, or chemically defined medium 105 to stimulate the production of an MSC-derived biologic component with pro-angiogenic factors 210. Examples of the xeno-free, and/or serum-free, or chemically defined medium are illustrated in Table 1 of the Examples. The biologic composition 220 is free of xeno- and serum-derived components, beneficially minimising the risk of potentially compromising the bioactivity of the composition 220 and thus positively impacting treatment efficacy. For example, animal serum components can have a high likelihood of being incorporated into a synthesized ECM resulting in possible undesired immunogenic responses.

The hypoxia-primed MSCs 205 cultured in a xeno-free, and/or serum-free, or chemically defined medium 105 synthesise the biologic component 210, a secretome 210 enriched in pro-angiogenic factors. In one example embodiment, the MSCs are cultured under 5% oxygen tension for 6 days without media changes to maximise the production of pro-angiogenic factors. As shown in FIG. 1 100, the MSC-derived biologic component 210 is aggregated and co-precipitated 110 with a negatively charged polymer 215 into a pericellular space of the hypoxia-primed MSC 205 to produce an aggregated biologic component 110. For example, the negatively charged polymer is dextran sulfate (DxS). In one example embodiment, the DxS is present at a concentration of 10 μg/ml during ECM synthesis. The negatively charged polymer 215 facilitates the deposition of an ECM with pro-angiogenic factors. In a most preferred example embodiment, the negatively charged polymer 215 is 500 kDA dextran sulfate sodium salt from Leuconostoc spp which promotes the deposition and retention of bioactive factors within the ECM for augmented bioactivity.

The MSCs 205 assemble the aggregated biologic component 110 and the negatively charged polymer 215, for example DxS, into an insoluble extracellular matrix (ECM) 115. The insoluble ECM is decellularized 230, 120 to obtain the biologic composition 220 comprising MicroParticles of Solidified Secretome (MIPSOS) 220, an ECM-DxS composite biologic 220. For example, the decellularization process comprises treatment with 0.15% (w/V) sodium deoxycholate supplemented with 0.175% (V/V) protease inhibitor cocktail, followed by DNase I treatment to remove residual DNA.

The ECM is processed by one or more chemical or mechanical means 125, for example mechanical collection, solubilization, and/or lyophilization. In an example embodiment, the processing comprises mechanical scraping, resuspension in deionized water, and lyophilization into fine powder form. This processing 125 advantageously allows for diverse application of the composition 220, for example as in a solid or liquid form.

The biologic composition 220 further includes a carrier component or a carrier means 130 to facilitate application of the biologic composition 220 at a target site, or a wound site. The carrier component is, for example, selected from hydrogels, sponges, sheets, powders, wound cleansers, antiseptic solutions, saline solution, topical antibiotics, barrier creams, emollients, hydrocolloid creams or dressings, foam dressings, antimicrobial dressings, collagen dressings, dressings with a foam and film layer, or combinations thereof. For example, the carrier component is a fibrinogen solution at a final concentration of 10 mg/ml in 0.9% NaCl, which is polymerised with thrombin at a final concentration of 5 U/ml to form a stable fibrin matrix for wound delivery. Beneficially furthering its application and versatility for different clinical application—the biologic composition 220 may be stored in a stabilized form and reconstituted or applied directly to a wound site as needed. In an example embodiment, the biologic composition retains bioactivity for several weeks to months when stored frozen and desiccated, providing a long-term therapeutic product.

The biologic composition 220 further comprises one or more bioactive factors. For example, the bioactive factors include one or more of: growth factors, cytokines, and chemokines. The bioactive factors include enhanced levels of vascular endothelial growth factor A (VEGF-A), particularly when synthesized under hypoxic conditions in R: Stem medium.

The biologic composition 220 of the claimed invention advantageously promotes angiogenesis in vitro and in vivo and accelerates re-epithelization and wound closure.

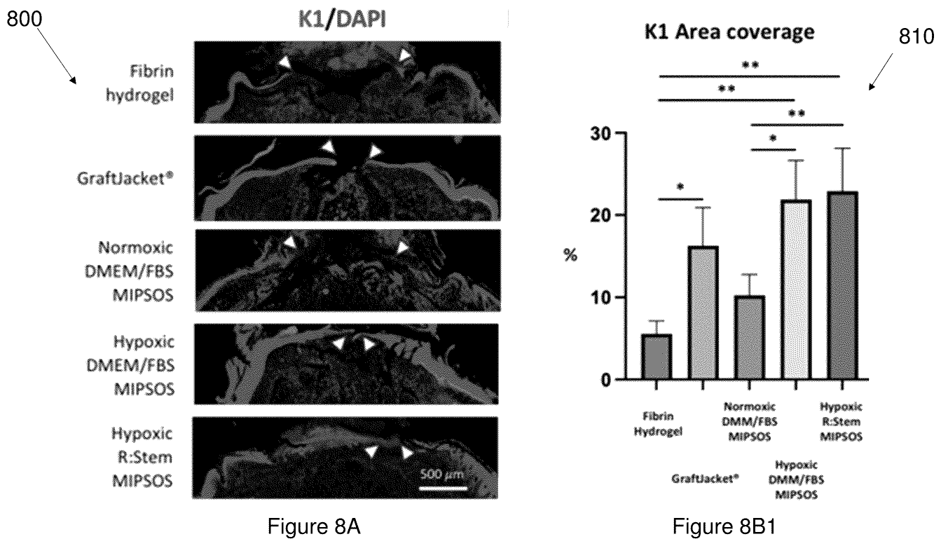

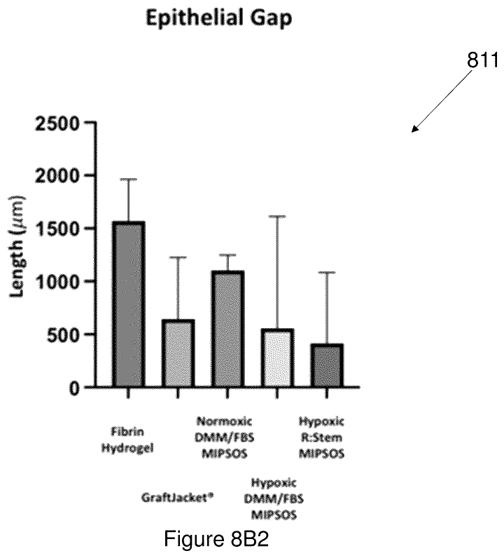

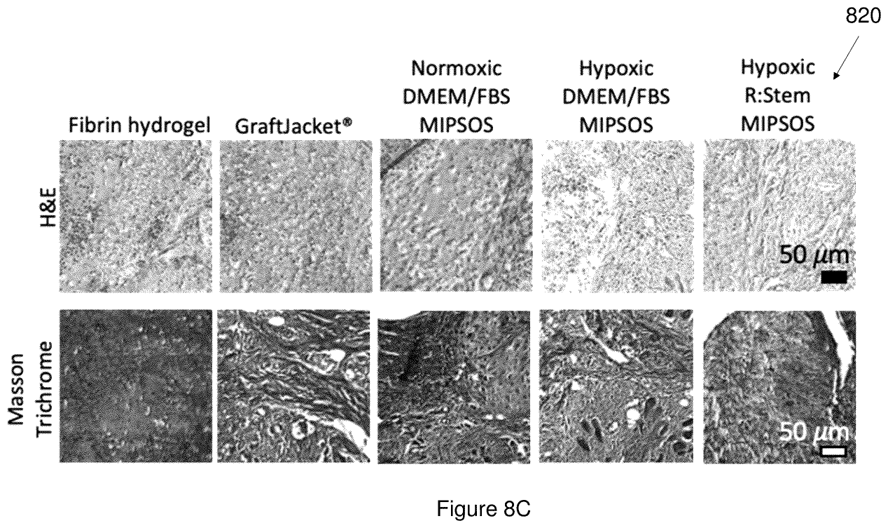

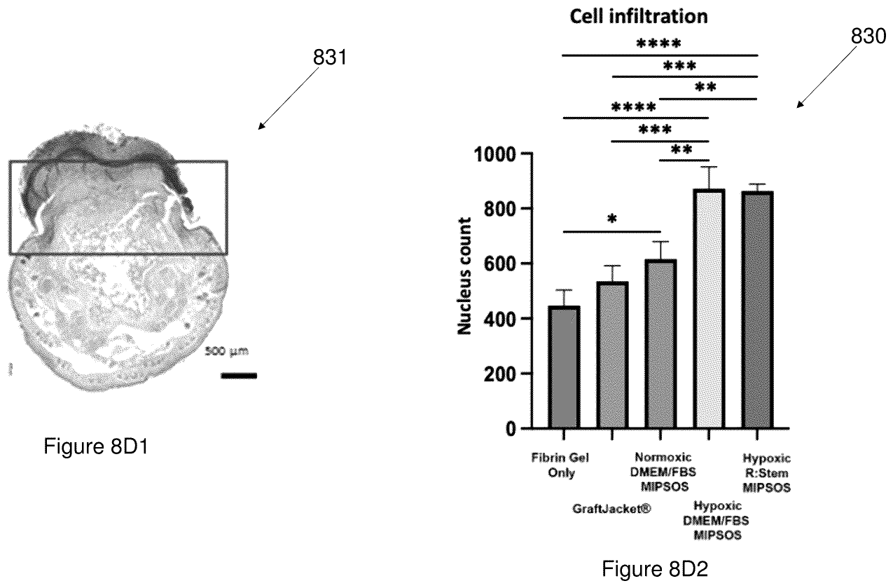

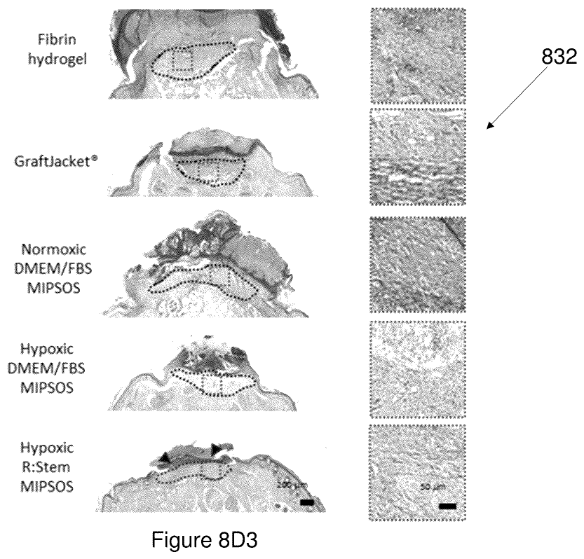

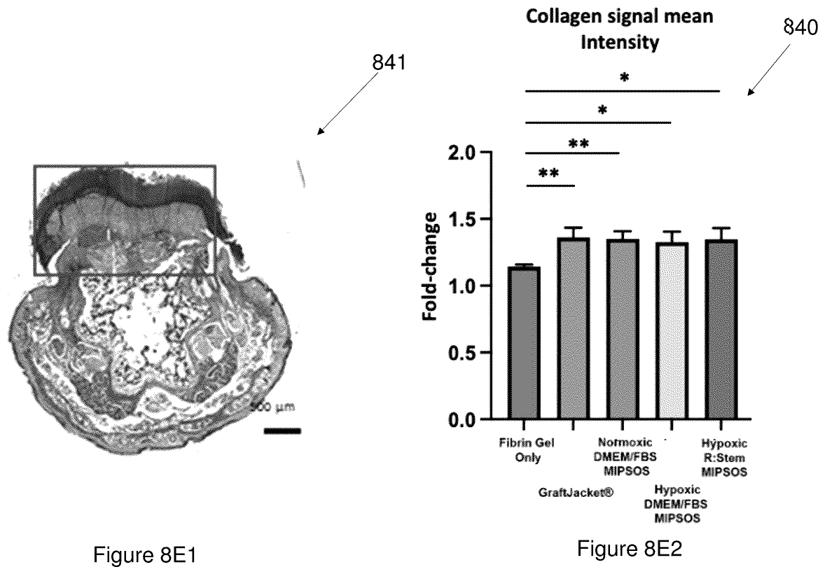

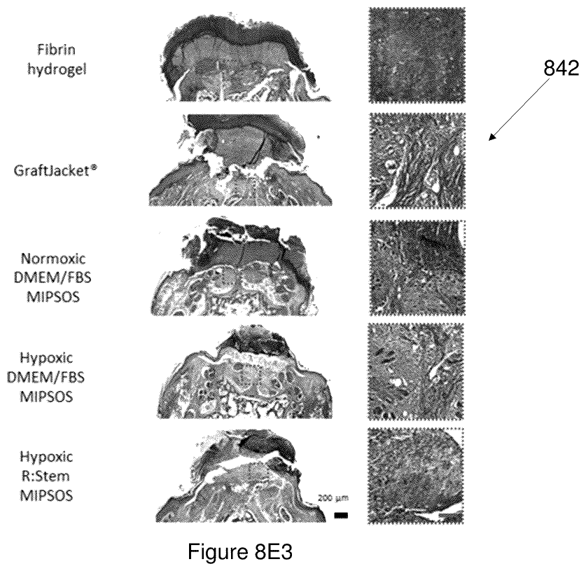

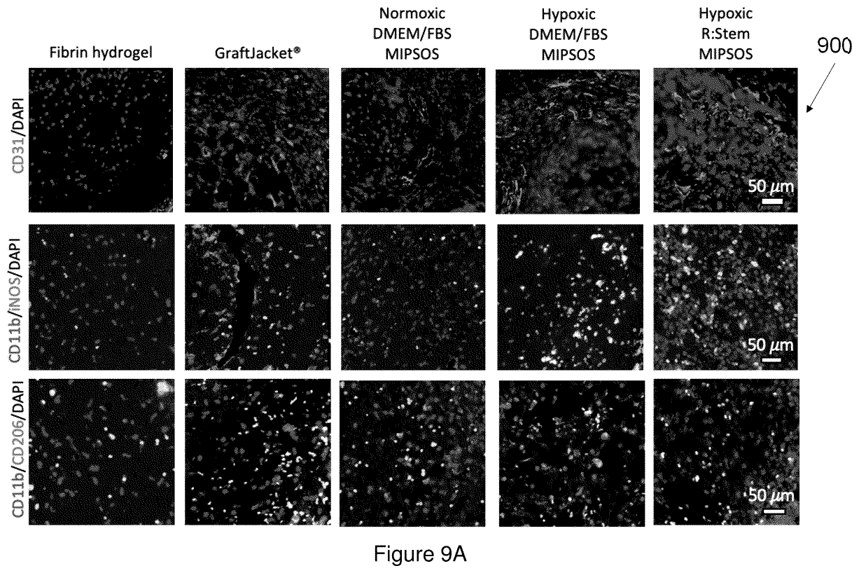

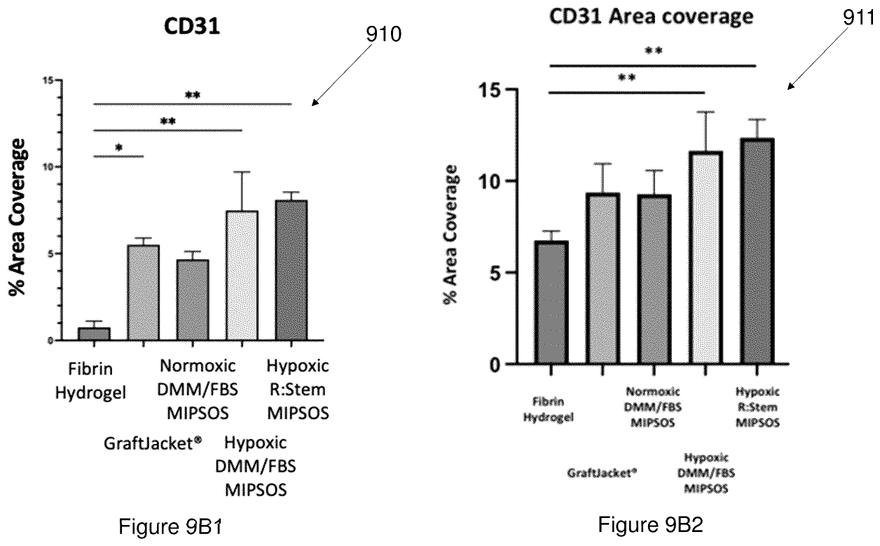

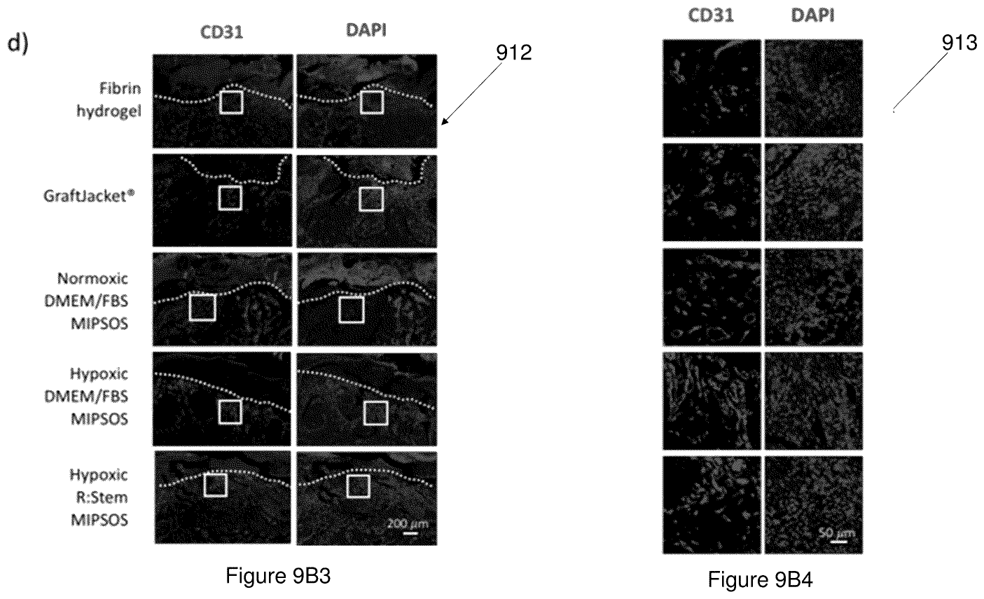

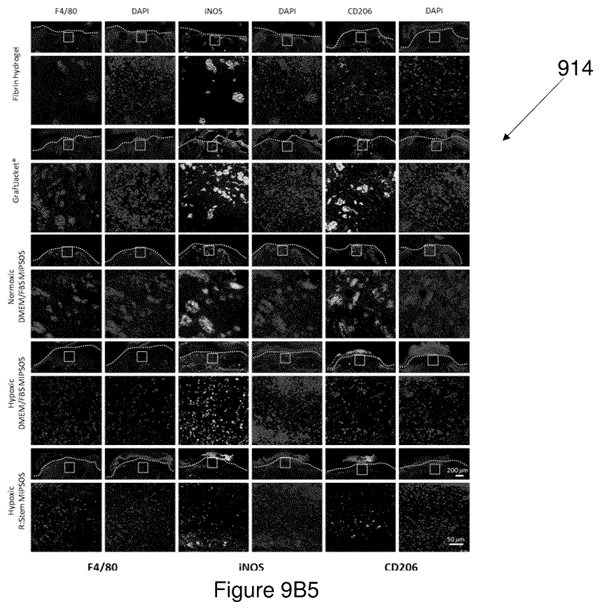

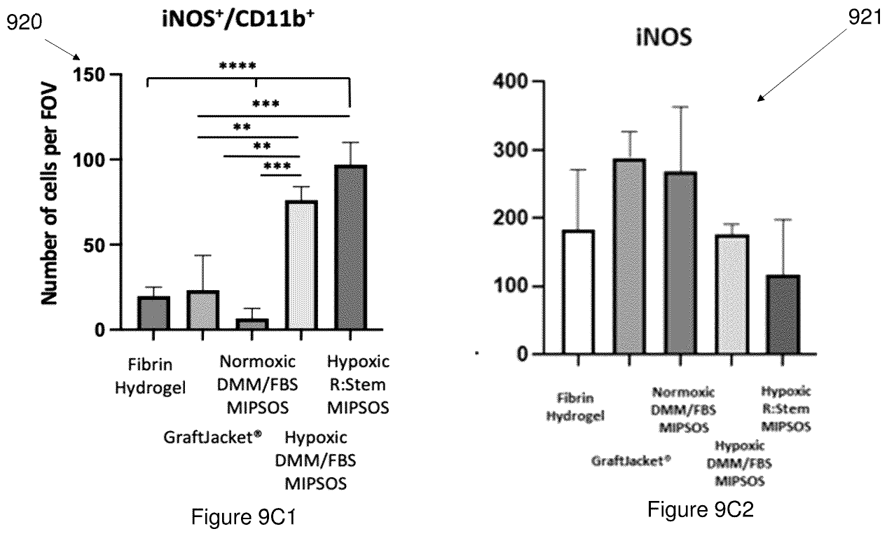

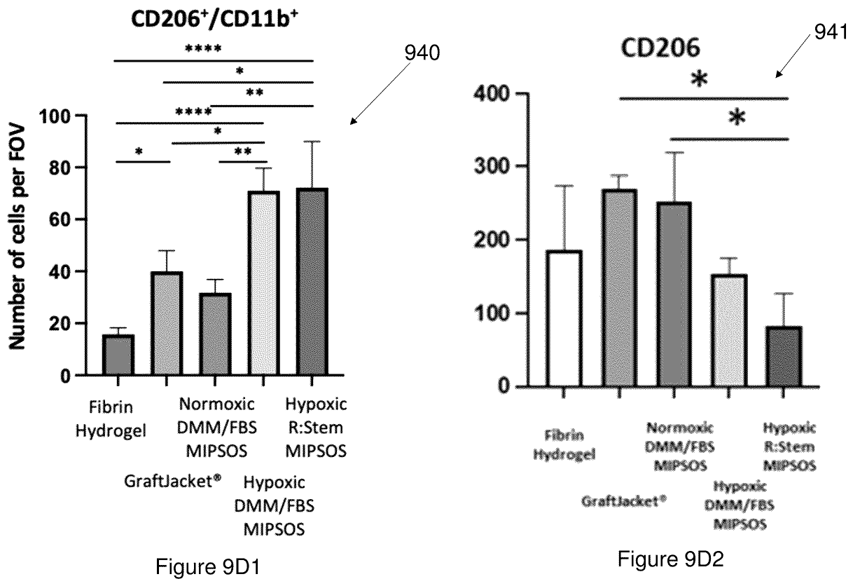

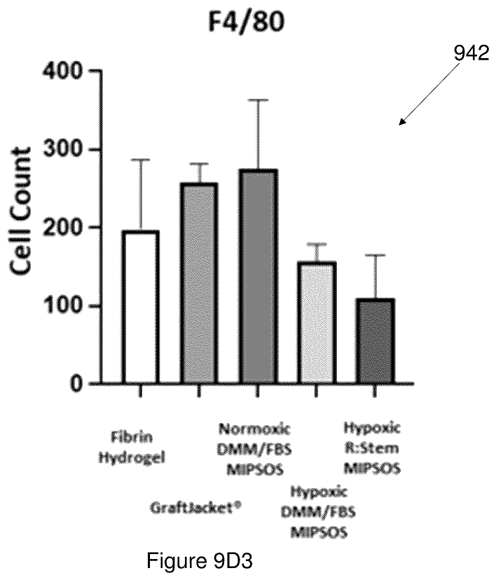

The biologic composition of the claimed invention demonstrates superior wound healing efficacy compared to clinically approved tissue-derived ECM products, with wound closure rates of up to 85% compared to 65% for GraftJacket® at 17 days in diabetic wound healing models, representing a 40% improvement in healing rate. The composition 220 promotes, for example, one or more of: wound closure, revascularization, cellular infiltration, ECM deposition, granulation tissue formation, and re-innervation with application in the treatment of wounds, including skin wounds, particularly diabetic skin wounds. The composition 220 demonstrates enhanced cellular infiltration, increased vessel density as measured by CD31 staining (FIG. 9B1 910, FIG. 9B2 911), and improved re-epithelialization (FIG. 8B2 811) as demonstrated by cytokeratin-1 expression. The composition 220 can be used in healing one or more of normal wounds, non-healing chronic wounds, chronic wounds, and diabetic wounds. In particular, the composition 220 is configured to enhance wound healing in wounds, for example diabetic wounds, by promoting angiogenesis, cellular infiltration, and granulation tissue formation. In an example embodiment, the composition accelerates wound closure by up to 40%.

Embodiments of the invention are also directed to a method for treating diabetic wounds, comprising administering to a wound a therapeutically effective amount of the biologic composition 220 as above wherein the composition 220 is applied topically to a wound site and accelerates wound re-epithelialization and closure.

Embodiments are also directed to a kit for the treatment of a wound, comprising the biologic composition 220 and instructions for use. The biologic composition 220 includes an applicator configured for application of the biologic composition 220 at a wound site, and the instructions for use include directions for the application of the biologic composition to enhance wound closure, cellular infiltration, and re-innervation in the wound.

The claimed invention beneficially provides an inexpensive, safe, and effective composition for promoting wound healing. The composition is versatile and can be tailored as needed, and accelerates wound healing by promoting angiogenesis. The composition is free of xeno- and serum-derived components thus avoiding immunogenic side effects while providing high therapeutic efficacy.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

The experiments as described below provide further examples of the invention as claimed as a highly effective, safe, and efficacious wound healing therapy.

### EXAMPLES

### Materials and Methods

### Cell Culture

Human BM-MSCs (Cat. No.: SCC034, Millipore Massachusetts, United States) and human umbilical vein endothelial cells (HUVECs) (Cat. No.: PCS-100-013, ATCC, Virginia, United States) were purchased from Millipore and Lonza, respectively. BM-MSCs were expanded and cultured in Dulbecco's Modified Eagle Medium (DMEM, with 1 g/L glucose and GlutaMAX) (Life Technologies, California, United States), supplemented with 10% Fetal Bovine Serum (FBS) (ExCell Bio, Shanghai, China) and 1% Penicillin/Streptomycin solution (Life Technologies, California, United States). HUVECs were expanded and cultured in EGM-2 BulletKit (Cat. No.: CC-3162, Lonza, Basel, Switzerland). Cells below passage 8 were used in all experiments.

### ECM-Based Material Synthesis

BM-MSCs were seeded at 7000 cells/cm² overnight to allow attachment. On the next day expansion medium was replaced with ECM induction media. All ECM induction media contains 10 μg/ml DxS (500 kDa, Cat. No.: D8906S, Sigma-Aldrich, St. Louis, United States) and 30 ug/ml ascorbic acid (Cat. No.: A8960, Sigma-Aldrich, St. Louis, United States) in various media listed in Table 1.

TABLE 1
Culture media utilized for MSC culture and ECM synthesis

| | | | | Chemically |
|---|---|---|---|---|
| Culture media | Abbreviation | Xenofree | Serum-free | Defined |
| Dulbecco's Modified Eagle | DMEM/FBS | X | X | X |
| Medium supplemented with | | | | |
| 0.5% Fetal Bovine Serum | | | | |
| CnT-Prime MSC Proliferation | CnT | ✓ | Low human- | |
| Medium, Xeno-Free (Cat. | | | serum | |
| No.: CnT-PR-MSC-XF, | | | | |
| CELLnTEC Advanced Cell | | | | |
| Systems AG, Bern, | | | | |
| Switzerland) | | | | |
| MSC NutriStem ® XF Medium | NutriStem | ✓ | Medium is | ✓ |
| (Cat. No.: 05-200-1A, | | | serum-free, | |
| Sartorius, Göttingen, | | | but supplement | |
| Germany) supplemented | | | contains human | |
| with PLTGold Human | | | platelet lysate | |
| Platelet Lysate, research | | | | |
| grade (Cat. No.: | | | | |
| PLTGOLD100R, Sartorius, | | | | |
| Göttingen, Germany) | | | | |
| Mesenchymal Stem Cell | Promocell | ✓ | ✓ | |
| Growth Medium XF (Cat. | | | | |
| No.: C-28019, PromoCell, | | | | |
| Heidelberg, Germany) | | | | |
| StemXVivo Xeno-Free | StemXVivo | | ✓ | |
| Human MSC Expansion | | | | |
| Media (Cat. No.: CCM021, | | | | |
| R&D Systems, Minnesota, | | | | |
| United States) | | | | |
| R: Stem (Cat. No.: EM1-500, | RStem | ✓ | ✓ | ✓ |
| Rohto, Osaka, Japan) | | | | |

Cells were cultured for 6 days, with no change of media, in separate incubators maintained at normoxia (21% O2), and hypoxia (5% O2), at 37° C., 5% CO2.

After 6 days of culture, cultures were either fixated or decellularised. Decellularisation was carried out by incubating the culture at room temperature for 15 mins with 0.15% (w/V) sodium deoxycholate (Cat. No.: 30970, Sigma-Aldrich, St. Louis, United States), supplemented with 0.175% (V/V) protease inhibitor cocktail (Cat. No.: P8340, Sigma-Aldrich, St. Louis, United States) in deionized water. Residual DNA was removed by subsequent incubation at 37° C. for 15 mins with 0.04 mg/ml DNAse I (Cat. No.: LS002007, Worthington Biochemical, Lakewood, United States) in 1× Dulbecco's Phosphate Buffered Saline with calcium chloride and magnesium chloride (Cat. No.: D1283, Sigma-Aldrich, St. Louis, United States). Resulting material was washed with deionized water, air dried and stored in 4° C. for short term or −20° C. for long term. Materials were left undisturbed on the culture vessels for all in vitro experiments. For in vivo experiments, the residual materials were removed mechanically with a cell scraper, resuspended in deionized water, and lyophilized into fine powders.

### Proliferation and Viability Assays

BM-MSC proliferation and viability was assessed by CCK8 assay and Live/Dead cell staining, respectively, according to manufacturer's protocols. In brief, the spent media were replaced with Cell Counting Kit 8 (Cat. No.: K1018, ApexBio, Houston, United States) supplemented with fresh growth medium. Absorbance of the medium was measured at 450 nm after 2 hours of incubation at 37′° C., 5% CO₂. Separate cultures were stained with LIVE/DEAD™ Viability/Cytotoxicity Kit for Mammalian Cells (Cat. No.: L3224, Invitrogen, Life technologies, United States), and imaged using an epifluorescence microscope (IX83, Olympus, Tokyo, Japan; ECLIPSE Ti2-A Imaging System, Nikon, Tokyo, Japan).

For HUEVC proliferation, cells were seeded onto the decellularized matrices at density of 7000 cells/cm² and cultured for 3 days at 37° C., 5% CO₂. Proliferation was then assayed as described above.

### Immunocytochemistry

All cultures were either fixed with ice-cold methanol for 20 minutes at −20° C., or with 4% Paraformaldehyde (PFA) for 30 mins at room temperature. Non-specific binding sites were blocked with 3% bovine serum albumin (BSA) in 1× Phosphate Buffer Saline (PBS), with or without 0.025% Triton X-100, for 1 hour at 4° C. The cultures were subsequently incubated with primary antibodies in 1% BSA in 1× PBS overnight at 4° C., followed by 2-hour incubation at room temperature with corresponding secondary antibodies in 1% BSA in 1× PBS. Images were taken using an epifluorescence microscope as described previously and analysed using ImageJ software. All antibodies and dyes used are listed in Table 2.

TABLE 2
Antibodies used for immunocytochemistry

| | | | |
|---|---|---|---|
| | | Catalog | |
| Host | Dilution | Number | Manufacturer |

Primary Antibodies

| | | | | |
|---|---|---|---|---|
| Col I | Mouse | 1:700 | C2456 | Sigma |
| Fibronectin | Rabbit | 1:500 | ab2413 | abcam |
| HIF-1-α | Rabbit | 1:200 | 20960-1-AP | Proteintech |
| VEGF-A | Mouse | 1:200 | 66828-1-Ig | Proteintech |
| Cytokeratin 1 | Mouse | 1:50 | ab9286 | abcam |
| (K1) | | | | |
| CD31 | Goat | 1:50 | AF3628 | R&D Systems |
| CD206 | Goat | 5 ug/ml | AF2535 | R&D Systems |
| iNOS | Rabbit | 1:20 | PA1-036 | Thermofisher |
| CD11b | Rat | 1:100 | MAB1124 | R&D Systems |
| TUBB3 | Rabbit | 1:100 | AC008 | Abclonal |

Secondary Antibodies

| | | | | |
|---|---|---|---|---|
| Anti-Rabbit 488 | Donkey | 1:500 (ICC) | ab150061 | abcam |
| | | 1:200 (IHC) | | |
| Anti-Mouse 488 | Goat | 1:500 (ICC) | ab150113 | abcam |
| Anti-Goat 488 | Donkey | 1:200 (IHC) | ab150129 | abcam |
| Anti-Mouse 555 | Goat | 1:500 (ICC) | ab150118 | abcam |
| | | 1:200 (IHC) | | |
| Anti-Rat 647 | Donkey | 1:200 (IHC) | ab150155 | abcam |

Probes

| | | | |
|---|---|---|---|
| Phalloidin-iFluor | 1:1000 | ab176756 | abcam |
| 555 | | | |
| DAPI | 1:1000 | 62247 | ThermoFisher |

(IF—immunofluorescence; Suppliers: Sigma-Aldrich, St. Louis, United States; Abcam, Cambridge, United Kingdom; Proteintech, Illinois, United States; Thermo Scientific ™, Massachusetts, United States)

Endothelial cell spheroid sprouting assay Endothelial cell spheroids were generated by seeding HUVECs into spheroid-forming plates (AggreWell™400, Cat. No.: 34415, Vancouver, Canada) at 700 cells/micro-well using fully supplemented EGM-2 medium and incubation at 37° C., 5% CO₂, overnight. Spheroids were collected in EGM-2 and then mixed 1 to 1 with EGM-2 containing Collagen I solution (Cat. No.: 5225, Advanced BioMatrix, California, United States) at final concentration of 1 mg/ml. The spheroid-collagen mixture was gently casted onto the matrices and the collagen hydrogel was allowed to polymerize for 2 hours at 37° C., after which fresh EGM-2 was overlayed. Spheroids were allowed to sprout for 2 days, before they were fixed with 4% PFA and stained for F-actin using the above mentioned Phalloidin-iFluor 555 probe. The spheroids were subsequently imaged using an epifluorescence microscope (ECLIPSE Ti2-A Imaging System, Nikon, Tokyo, Japan) and their sprout length determined using ImageJ software.

### Gel Electrophoresis and Silver Staining

Equal volume of extracts was heated with 5% (V/V) of 2-mercaptoethanol for 5 minutes at 95° C., before being resolved in NuPAGE™ 4 to 12%, Bis-Tris gels (NP0322BOX, Life Technologies, Invitrogen™, California, United States) at 200V in 3-(N-morpholino) propanesulfonic acid (MOPS) Buffer (Cat. No.: NP0001, Life Technologies, Invitrogen™, California, United States).

Protein bands were stained with Pierce™ Silver Stain Kit (Cat. No.: 24612, Life Technologies, California, United States), according to manufacturer's protocol. Gels were imaged using Bio-Rad ChemiDoc Imaging System (Bio-rad, California, United States), and analyzed with Image Lab Software v6.0.1 (Bio-rad, California, United States).

### Tail Skin Wound Model

All animal experiment procedures were approved by the Animal Experimentation Ethics Committee, The Chinese University of Hong Kong. 6-8 weeks old male db/db mice (n=8 per condition) were used. Mice were housed in acryl cages in a light-controlled room with a 12:12 hour light/dark cycle with ad libitum access to water and rodent chow.

For creation of the wound, mice were anaesthetized using continuous isoflurane. Following negative paw reflex confirming deep sedation, two 1 cm×0.3 cm spaced 0.5 cm apart full thickness wounds were created on the tails of mice. Thrombin (Cat. No.: 605190, Sigma-Aldrich, St. Louis, United States)-cross linked fibrinogen (Cat. No.: 341576, Sigma-Aldrich, St. Louis, United States) solution was utilized as a delivery vehicle. For this, cell-derived ECM-based microparticles, prepared as described above and GraftJacket® (Wright Medical Group, Inc., United States) ground into microfragments, were suspended at a final concentration of 10 mg/ml in fibrinogen solution (final concentration 10 mg/ml in 0.9% NaCl). Briefly before topical application into wounds, fibrinogen solutions were supplemented with thrombin (final concentration 5 U/ml) to facilitate formation of a stable fibrin matrix within the wound. The wounds were covered with transparent film dressing.

The size of the wounds were monitored by imaging the wounds at fixed distance across different time points, and wound areas were subsequently quantified using ImageJ software. Rate of healing of each wound on each mouse was calculated using simple regression analysis model by GraphPad Prism v10.0 (GraphPad Software, California, United States).

### Histology and Immunohistochemistry

Whole tails of the mice were harvested at the end of the experiment, after animals were euthanized by carbon dioxide. Tails were fixed with 4% PFA for 1 day, before being transferred into 14% Ethylenediaminetetraacetic acid (EDTA) solutions, Tails in EDTA were incubated at 37° C. for 7 days, with daily changes of EDTA solution, to achieve decalcification of the tail bone. Resulting tissues were embedded in paraffin and 10 μm sections were prepared. Sections were stained with hematoxylin & eosin (H&E), masson's trichrome and immunostained for markers listed below. Images were taken using an epifluorescence microscope as described previously and analysed using ImageJ software.

In brief, H&E images were first deconvoluted using the ImageJ color deconvolulation2 plugin tool, nuclei were then analysed and counted using the analyse particle function of ImageJ. Mason Trichrome images were analysed using the histogram function of ImageJ to extract the mean intensity of the blue signal.

### Statistical Analysis

All statistical analysis was performed using one- or two-way ANOVA assuming equal variance with GraphPad Prism v10.0 (GraphPad Software, California, United States) with p values less than 0.05 considered as statistically significant. All in vitro experiments were performed at least as three biologically independent repeats, while 8 animals per condition were used for in vivo experiments.

### Results

### Example 1: Biologic Composition (Mipsos) can be Synthesised Under Xeno- and/or Serum-Free Conditions

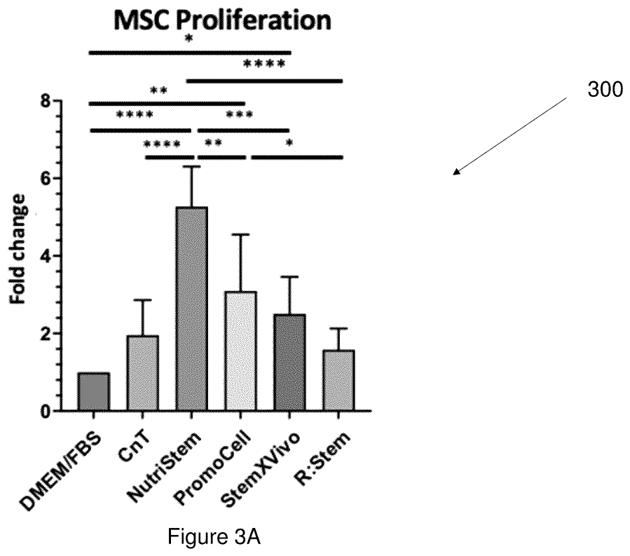

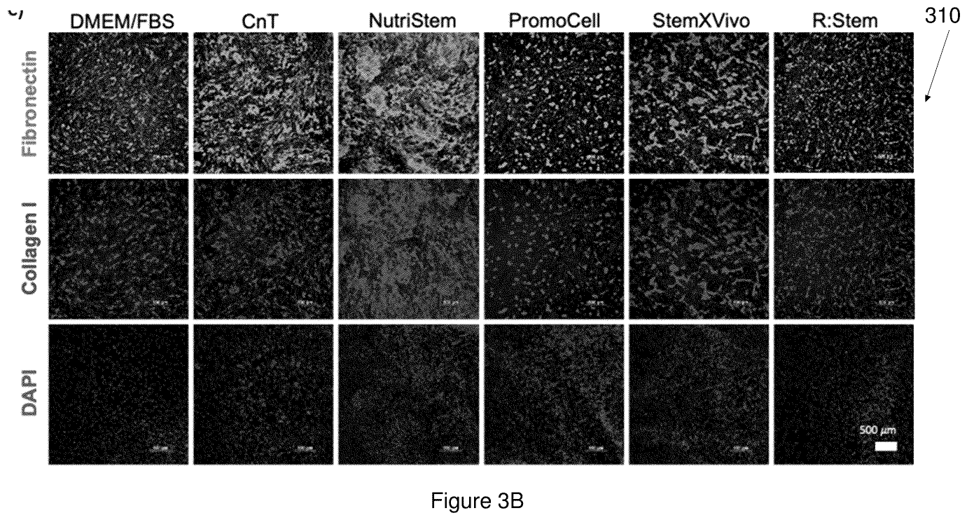

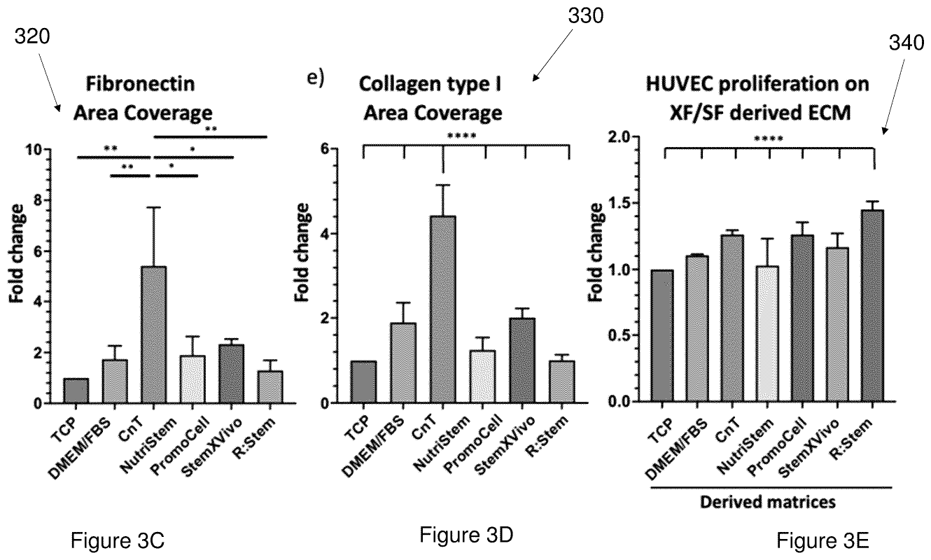

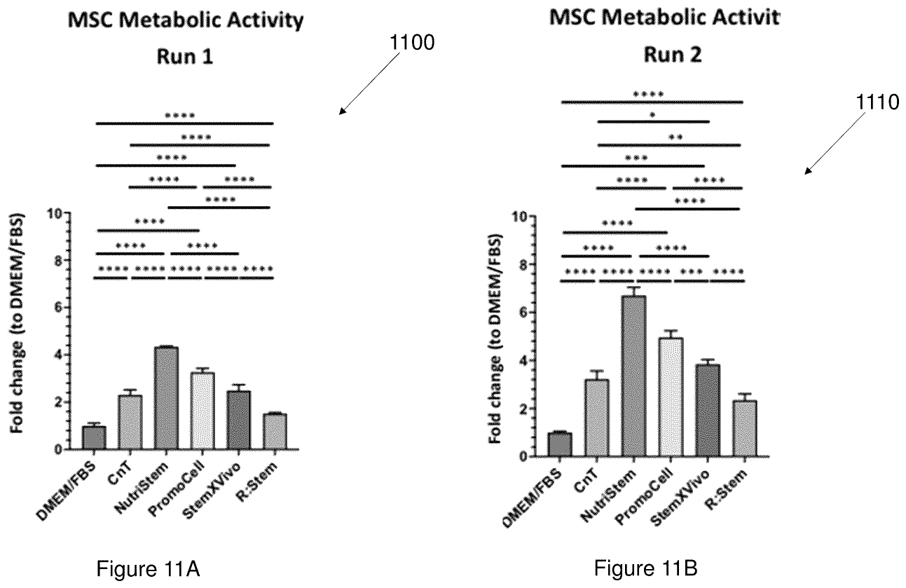

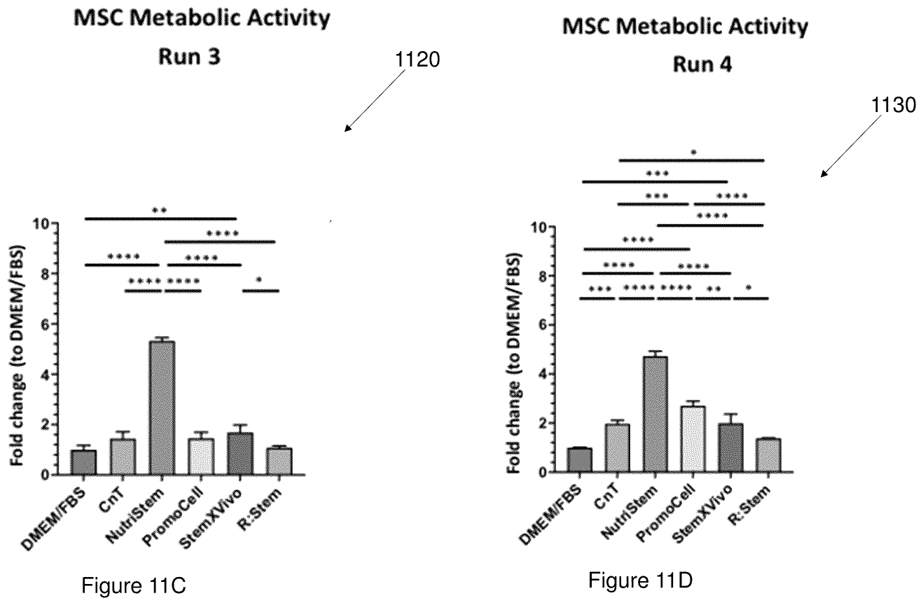

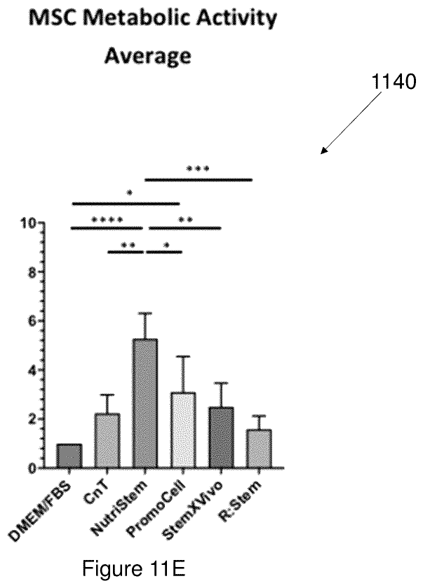

In order to produce MIPSOS under xeno- and/or serum-free conditions, BM-MSCs were cultured in several commercially available xeno-free (XF) and/or serum-free (SF) media (as shown previously in Table 1) in the presence of DxS and ascorbate at previously established concentrations for 6 days (FIG. 2 200). All media supported MSC proliferation over the time period of ECM synthesis. Indeed, cell numbers in the chosen media enhanced MSC proliferation resulting in 2 to 5-fold higher cell yields after 6 days (FIG. 3A 300, FIGS. 11A-11E 1100, 1110, 1120, 1130, 1140).

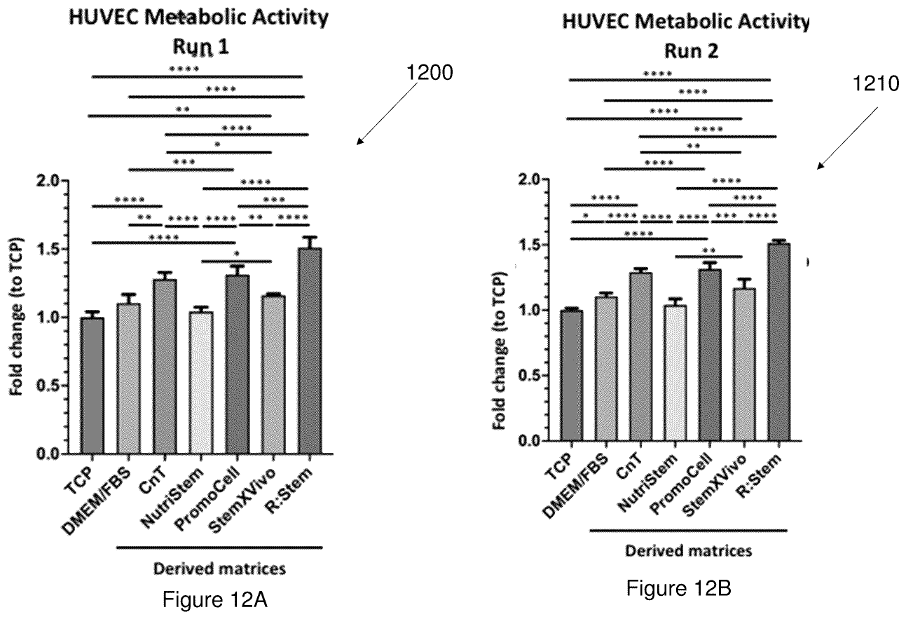

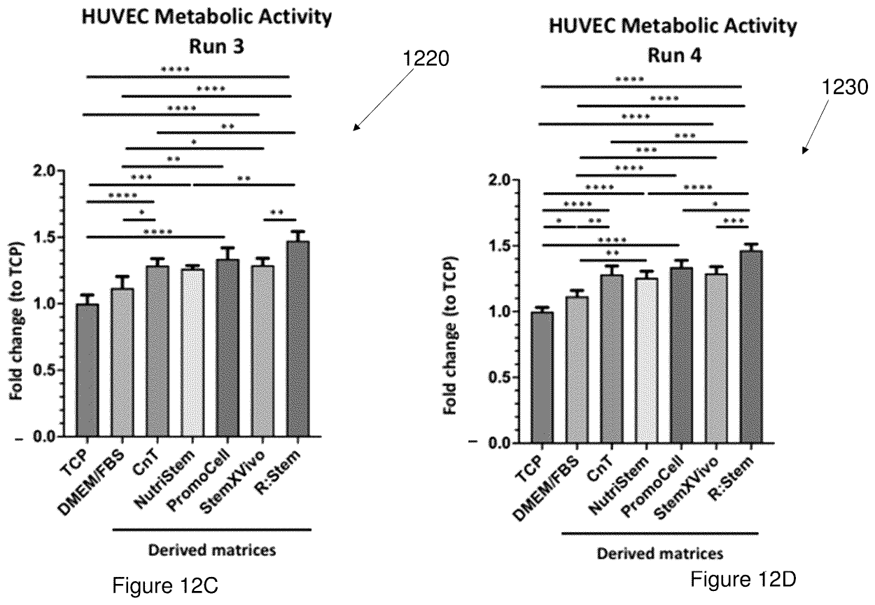

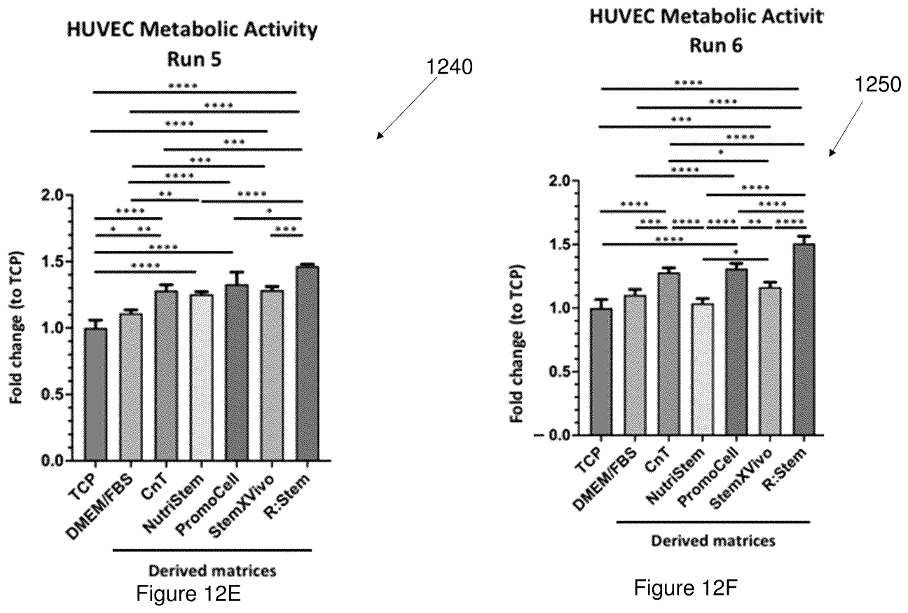

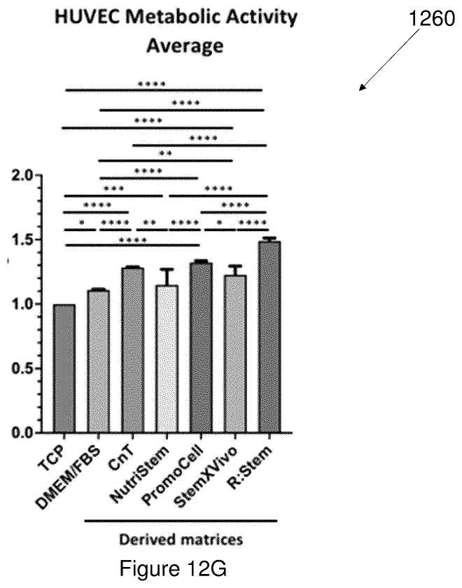

As shown in FIGS. 12A-12G (1200, 1210, 1220, 1230, 1240, 1250, 1260) HUVECs were seeded on decellularized matrices and cell metabolic activity was determined by CCK8 assay after 3 days. All matrices synthesized in the presence of XF/SF media promoted HUVEC metabolic activity, illustrating enhanced cell proliferation.

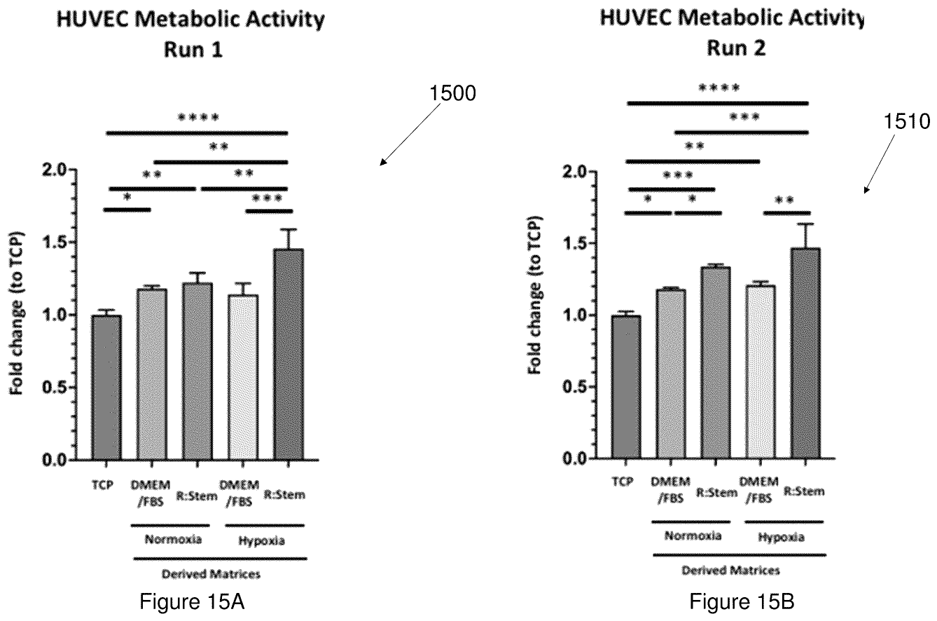

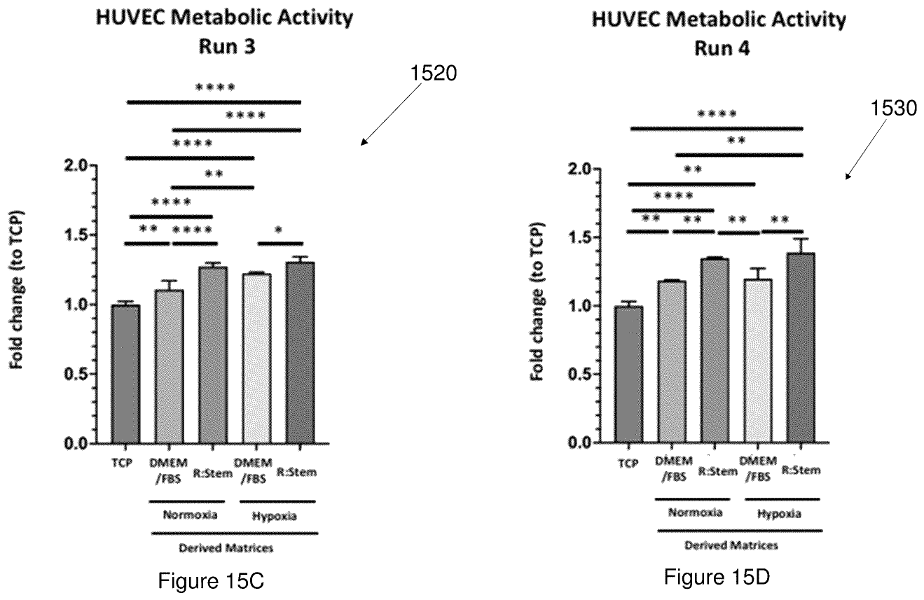

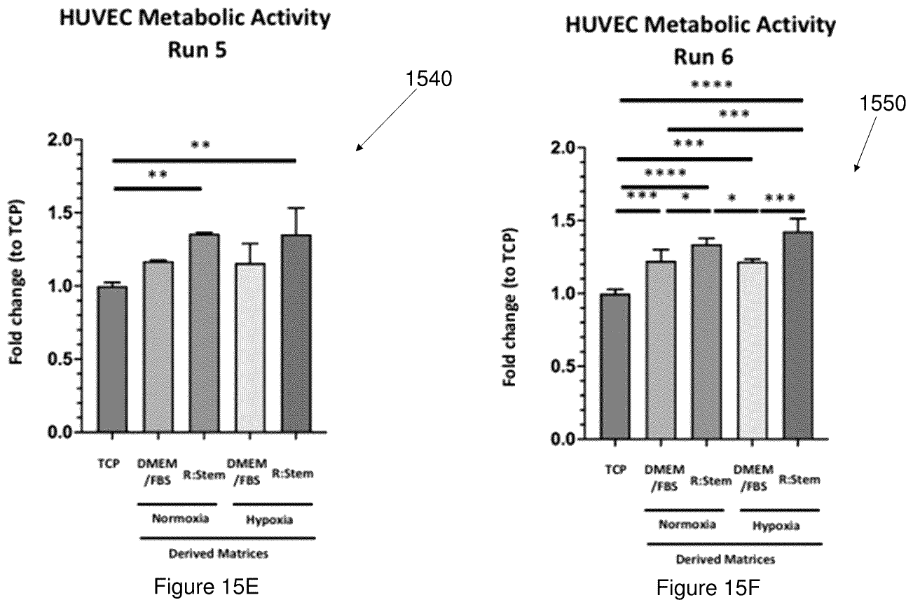

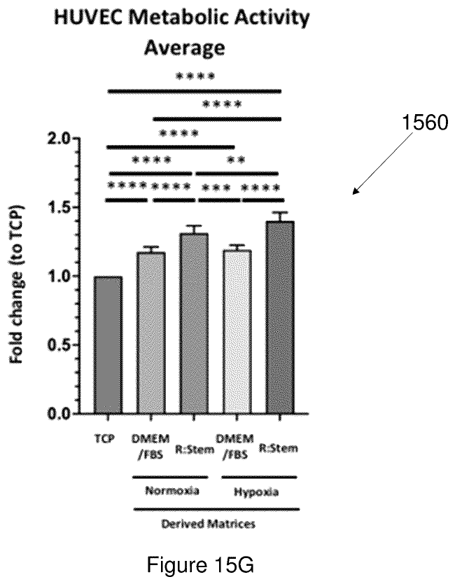

In order to determine whether the amount of deposited ECM was affected by the various media, MSC cultures were stained for major ECM structural components collagen I and fibronectin and the stained area was quantified. Culturing MSCs in NutriStem medium significantly promoted the deposition of both ECM components, whereas cells cultured in other media exhibited comparable amounts of deposited collagen I and fibronectin as in DMEM/FBS cultures (FIG. 3B-3D). In fact, quantities of collagen I and fibronectin exhibited similar trends as was noted for MSC numbers in the various media (FIG. 3A-3D 300, 310, 320, 330). Noteworthy, ECM components in all cultures exhibited a granular pattern, as previously reported for ECM deposited in the presence of DxS. Upon successful decellularization (FIG. 15A-15G 1500, 1510, 1520, 1530, 1540, 1550, 1560), the resulting matrices were evaluated for their pro-angiogenic properties by examining their ability to support endothelial cell proliferation. For this, HUVECs were seeded on decellularized matrices and cell numbers were determined by CCK8 assay after 3 days. All matrices synthesized in the presence of XF/SF media promoted HUVEC proliferation, exceeding endothelial cell numbers derived from cultures on tissue culture polystyrene (TCP). Remarkably, HUVECs cultured on matrices synthesized in the R: Stem medium exhibited the strongest potential to promote endothelial cell proliferation (FIG. 3E 340). These results were unexpected, as R: Stem medium did not promote ECM deposition as effectively as the other XF/SF media (FIG. 3B-3D 310, 320, 330), suggesting that amounts of deposited collagen I and fibronectin did not necessarily correlate with the extent of pro-angiogenic bioactivity of the biologic. Taken into consideration that although R: Stem did not promote ECM deposition, the R: Stem-derived matrices exhibited the strongest pro-angiogenic potential, R: Stem was chosen for further experiments (FIGS. 15A-15G 1500, 1510, 1520, 1530, 1540, 1550, 1560).

### Example 2: Hypoxia and R: Stem Medium Synergistically Augment Pro-Angiogenic Properties of Biologic Composition (Synthesized Mipsos)

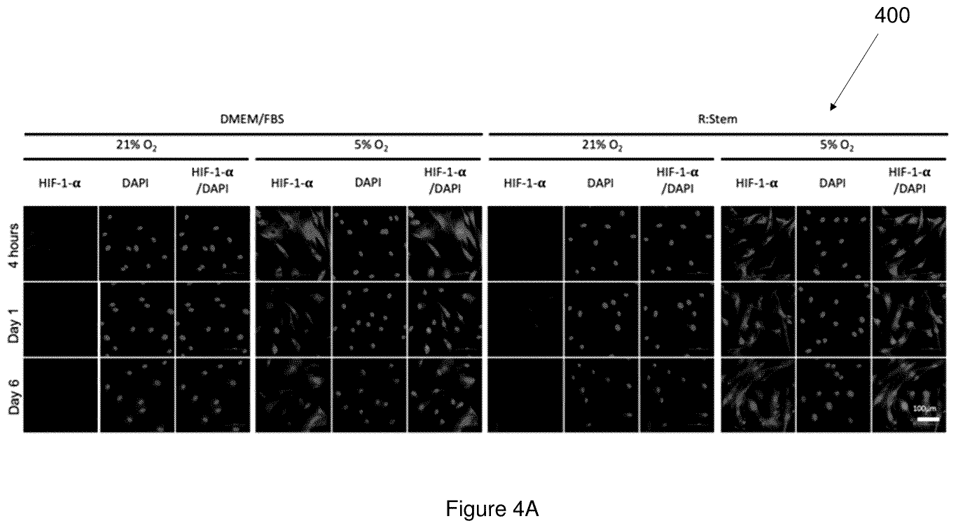

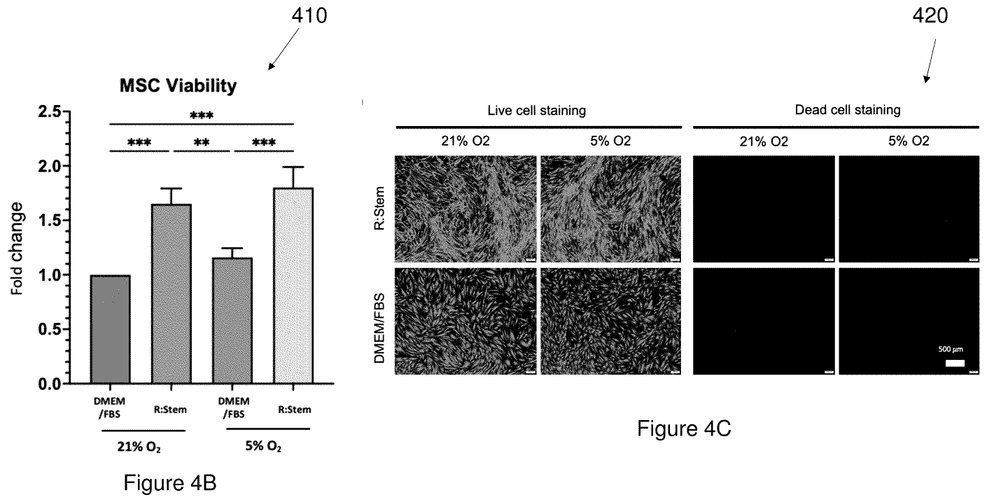

Culturing MSCs under hypoxic conditions was reported to protect transcription factor hypoxia-inducible factor 1-alpha (HIF-1-α) from degradation, facilitating its translocation to the nucleus, thereby driving the transcription of angiogenesis-related genes. This would in turn result in an increased secretion of pro-angiogenic factors which the inventors anticipated to be incorporated into their ECM during assembly and thus hypothesized that the resulting MIPSOS assembled under hypoxic conditions would enhance the pro-angiogenic potential of the biologic composition. Successful stabilisation of HIF-1-α was confirmed in MSCs cultured under low oxygen tension, independent of culture medium utilized, throughout the whole period of ECM deposition of 6 days (FIG. 4A 400). MSC cultures exhibited significantly increased cell numbers, when grown in R: Stem (FIG. 11A-11E 1100, 1110, 1120, 1130, 1140), while hypoxic cell culture conditions further elevated these cell numbers for both cell culture media (FIG. 4B 410). Live/Dead cell staining confirmed that increased cell numbers were caused by enhanced cell proliferation, as no cell death was observed in any of the cultures (FIG. 4C 420). Hence, chosen cell culture conditions had no adverse effects on MSC cultures.

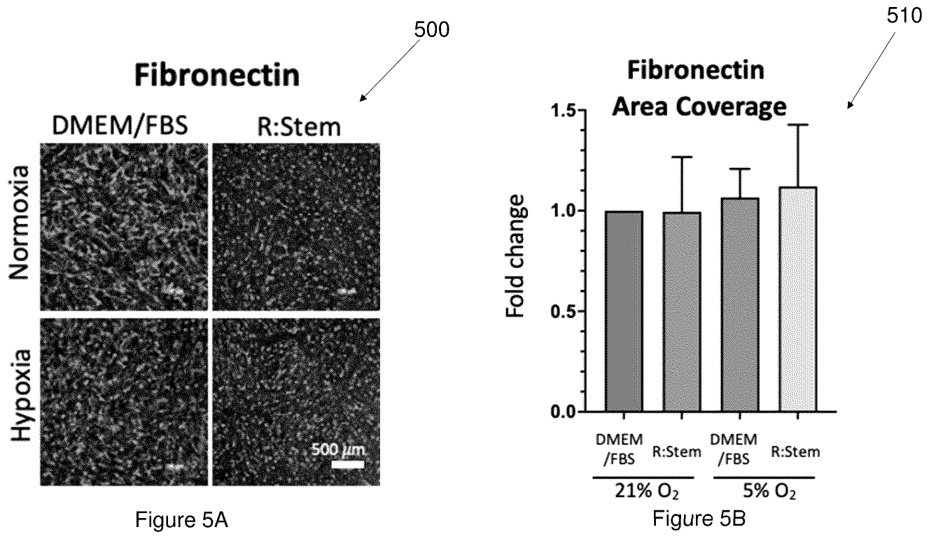

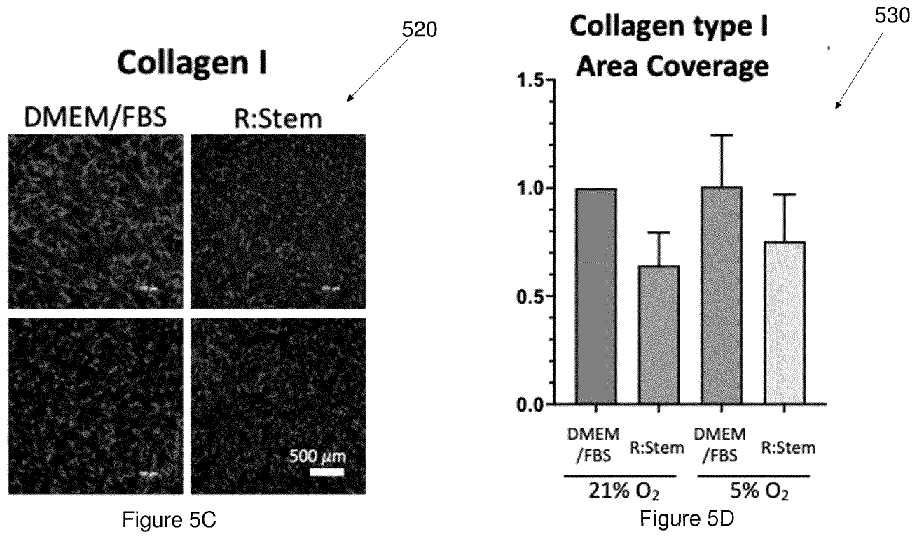

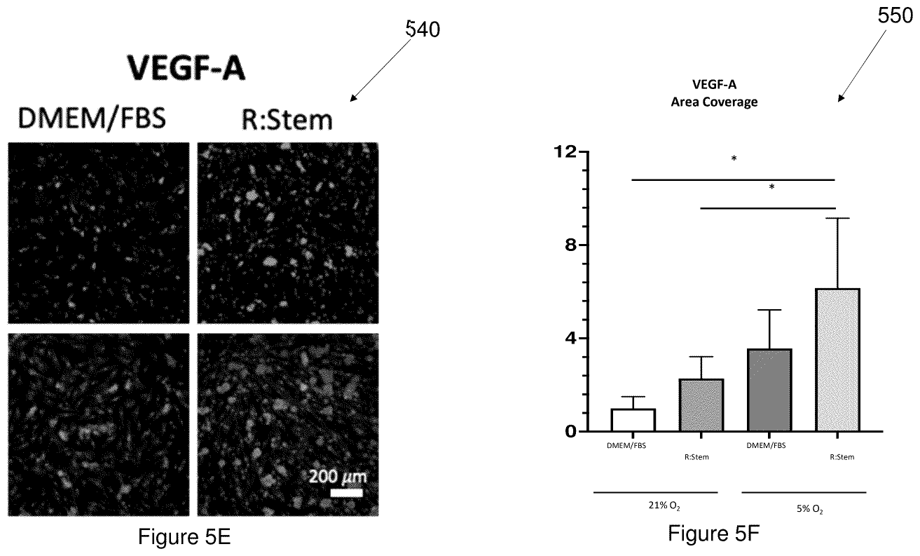

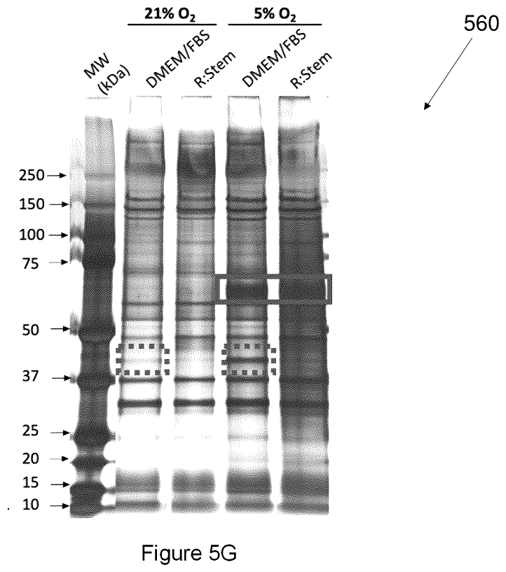

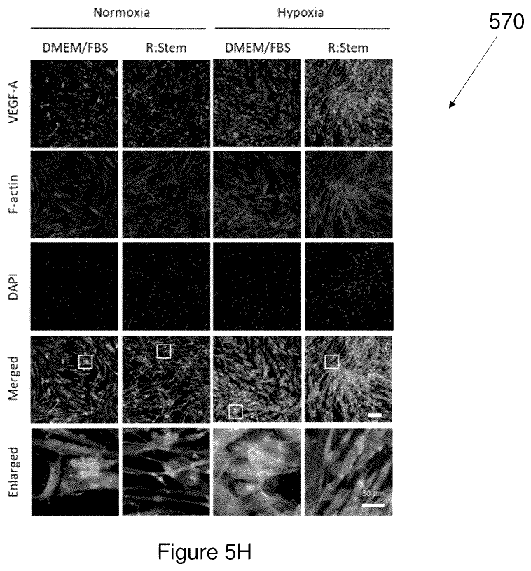

Upon successful establishment of hypoxic cultures, the effect of hypoxia on ECM deposition and composition was evaluated. Staining for vascular endothelial growth factor A (VEGF-A), a downstream target of HIF-1-α activation, revealed an elevated VEGF synthesis in MSCs cultured in R: Stem as compared to DMEM/FBS, which was further enhanced by hypoxic conditions for both media types (FIGS. 5E 540 and 5F 550). The inventors found that VEGF-A staining exhibited a granular pattern in all cultures, demonstrating the successful incorporation of this ECM-bound factor into the DxS-enhanced matrix, while in R: Stem cultures additional VEGF-A positive staining of cell bodies was observed (FIG. 5H 570, FIGS. 5E 540, and 5F 550). Gross molecular profiling of decellularised matrices by gradient PAGE further revealed a similar protein band pattern for all samples, with a prominent protein band at approximately 60 kDa found in matrices synthesised under hypoxic conditions and a 40 kDa protein band more prominent in matrices synthesized using DMEM/FBS (FIG. 5G 560).

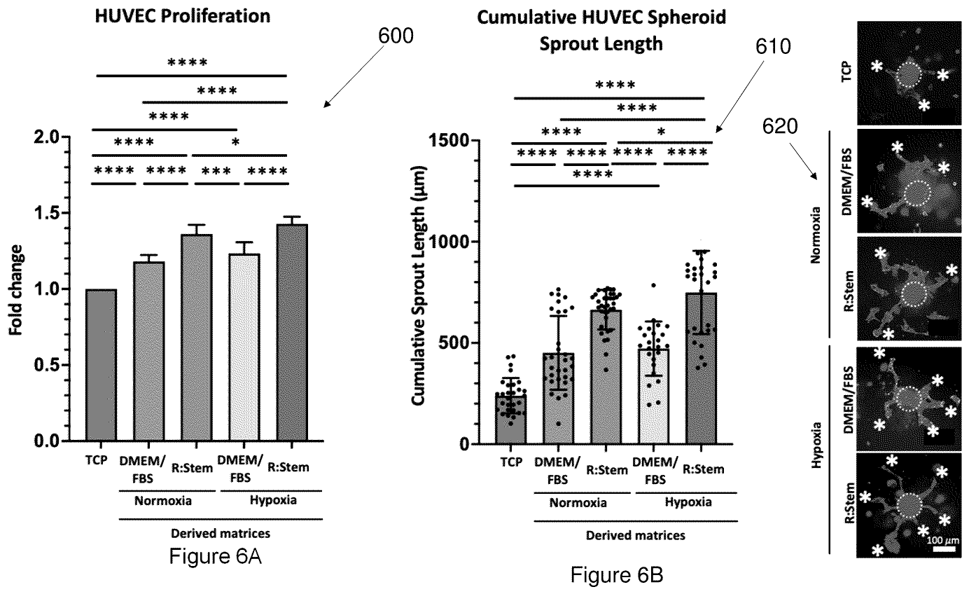

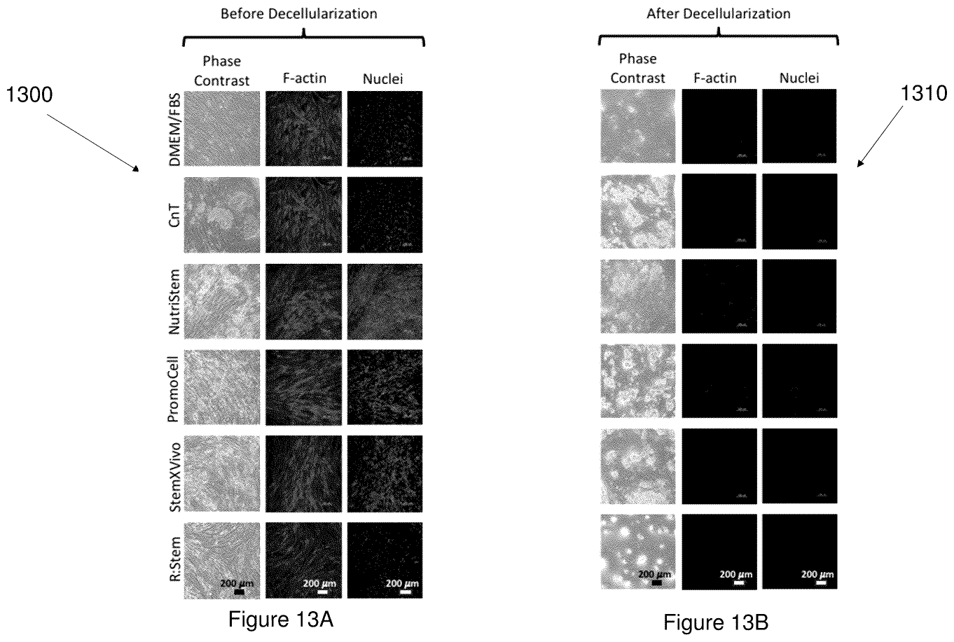

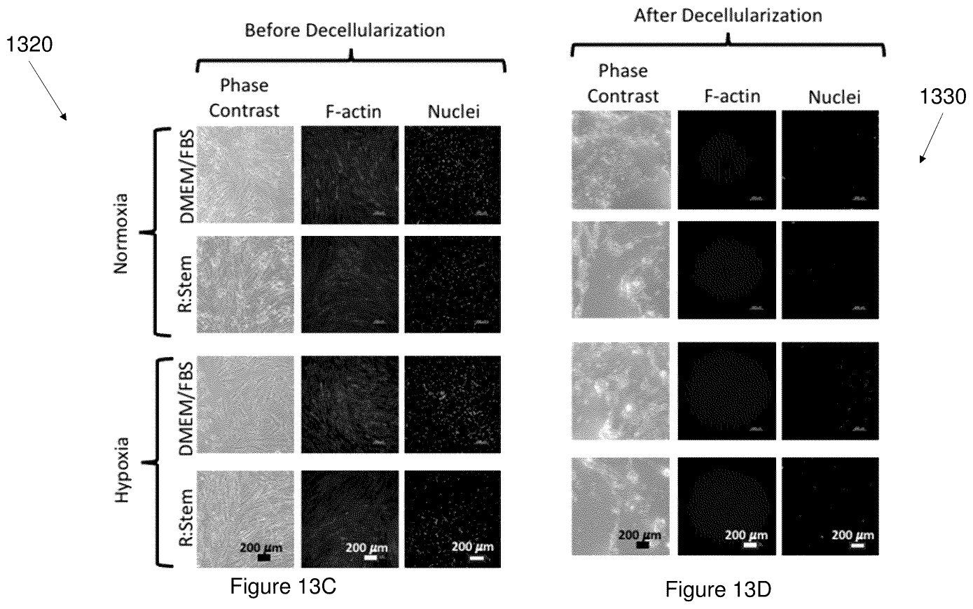

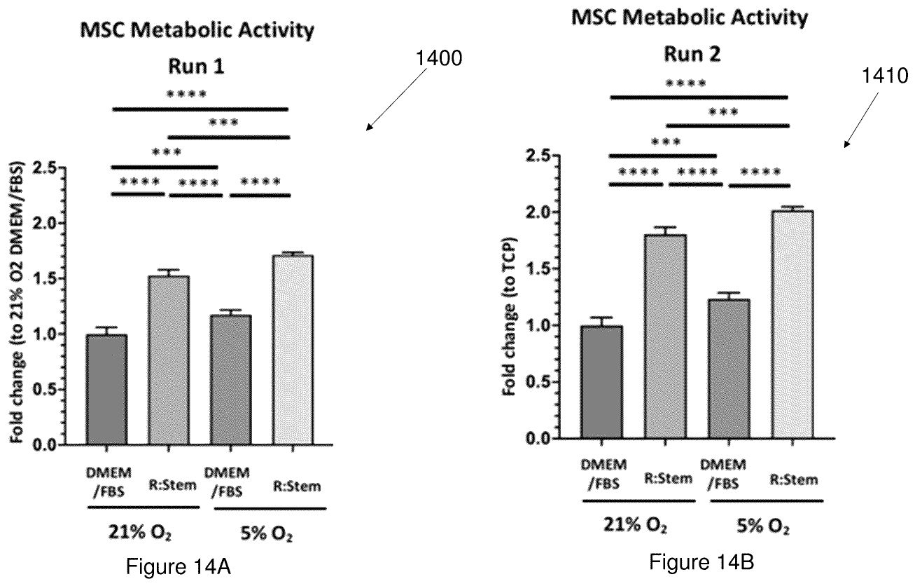

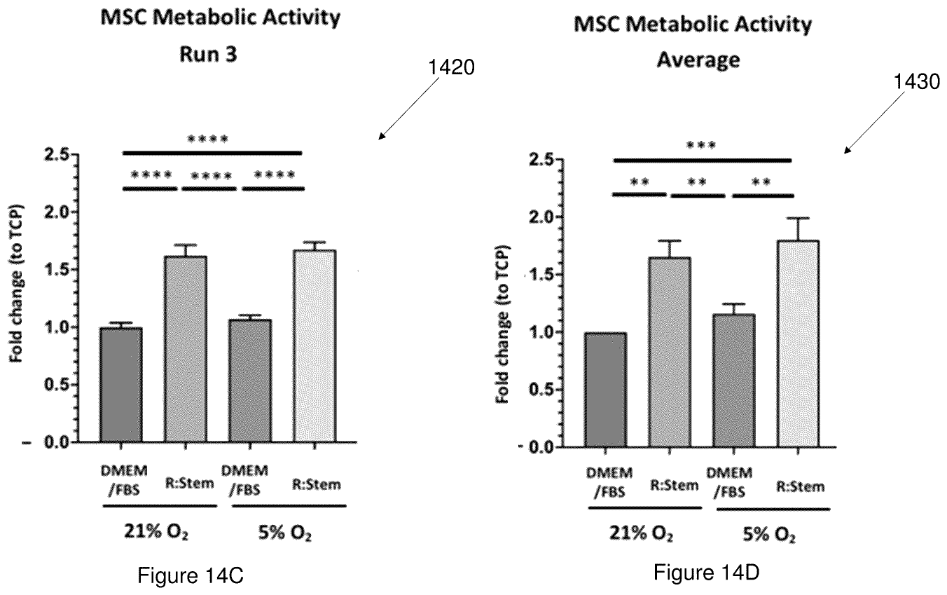

When decellularised matrices (FIG. 13A 1300) were seeded with HUVECs, all materials significantly promoted endothelial metabolic activity, as compared to TCP, suggesting enhanced proliferation. As expected, both DMEM/FBS and R: Stem-derived ECM-based materials synthesized under hypoxia were able to significantly promote endothelial metabolic activity as compared to their respective counterparts synthesised under normoxic conditions (FIG. 6A 600, FIG. 13B 1310). Interestingly, materials synthesized using R: Stem under normoxia promoted endothelial metabolic activity more efficiently than matrices assembled in DMEM/FBS and under hypoxia, suggesting that choice of medium is of high relevance, when synthesising ECM-based materials with specific bioactivities. When endothelial spheroids were seeded directly onto decellularised matrices in collagen I hydrogels, a significantly longer cumulative sprout length per spheroid was observed on all matrices as compared with TCP, with the most significant increase in cumulative sprout length on matrices assembled in R: Stem and under hypoxia. Again, the medium of choice for ECM assembly had the most prominent effect on the pro-angiogenic potential of matrices, as matrices synthesized in R: Stem outperformed matrices assembled in DMEM/FBS (FIG. 6B 610, 620).

The inventors found that when decellularised matrices were seeded with HUVECs, all materials significantly promoted endothelial proliferation, as compared to TCP. Advantageously, both DMEM/FBS and R: Stem derived ECM-based materials were able to significantly promote endothelial proliferation (FIG. 6A 600). Materials synthesised using R: Stem under normoxia promoted endothelial proliferation. When endothelial spheroids were allowed to sprout in collagen I hydrogels overlaying the various ECM-based substrates, a significantly longer cumulative sprout length per spheroid was observed on all matrices, with significant increase in cumulative sprout length on matrices assembled in R: Stem under hypoxia. Different mediums for ECM assembly influenced the pro-angiogenic potential of matrices. (FIG. 6B 610).

### Example 3: Biologic Composition (Mipsos) Synthesised Under Defined Conditions and Under Hypoxia Accelerated Diabetic Wound Healing in a Mouse Tail Wound Model

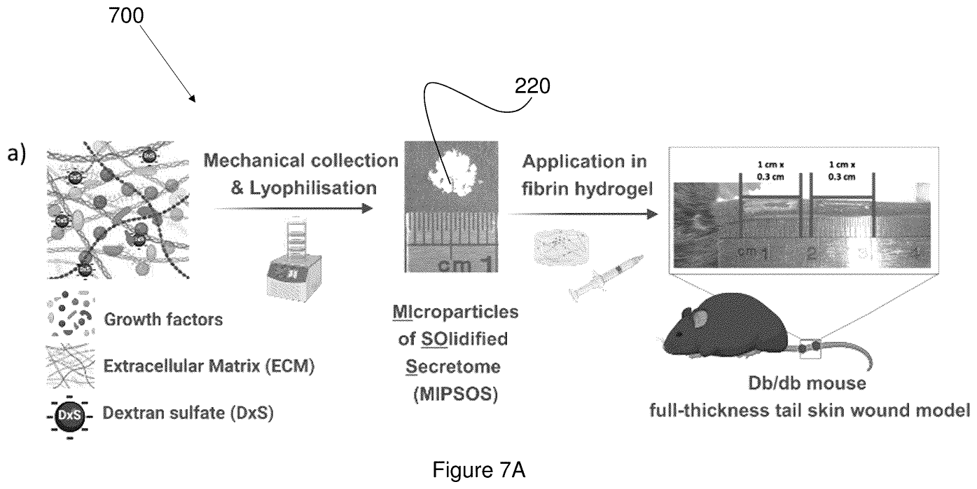

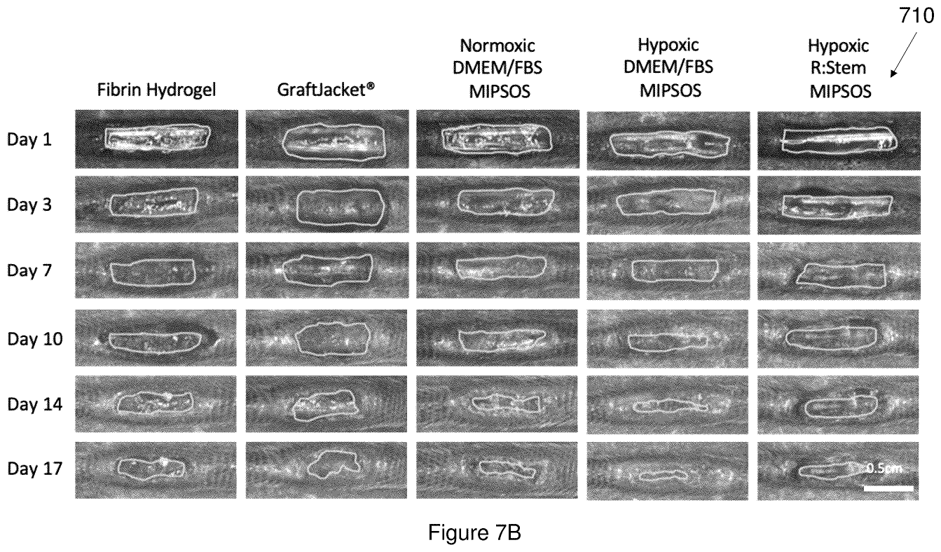

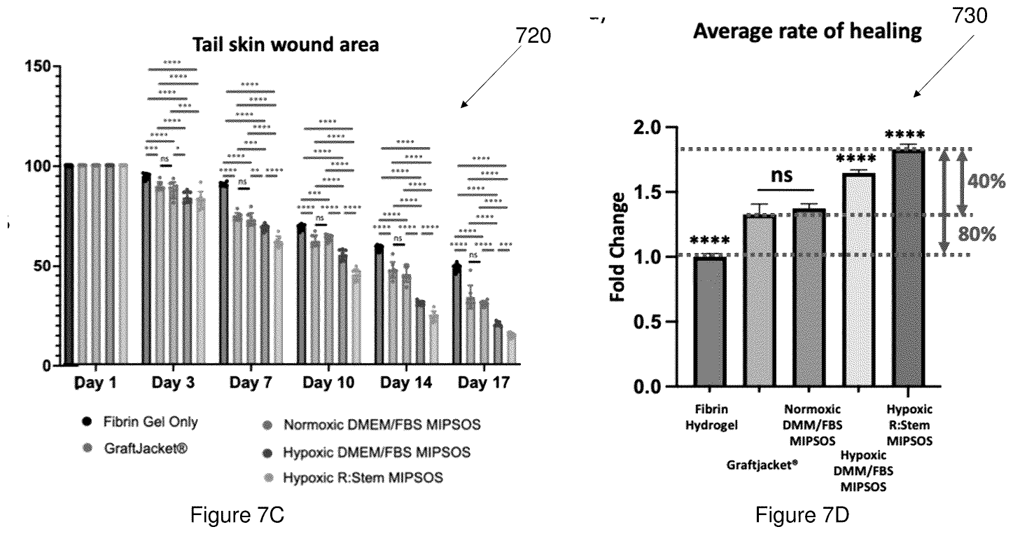

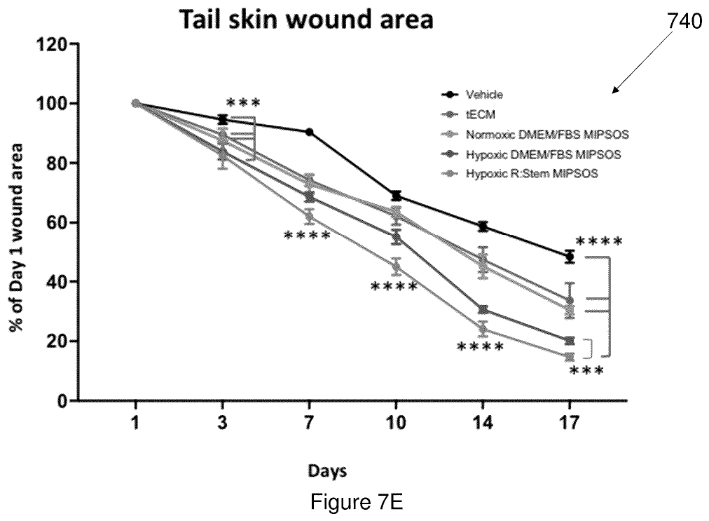

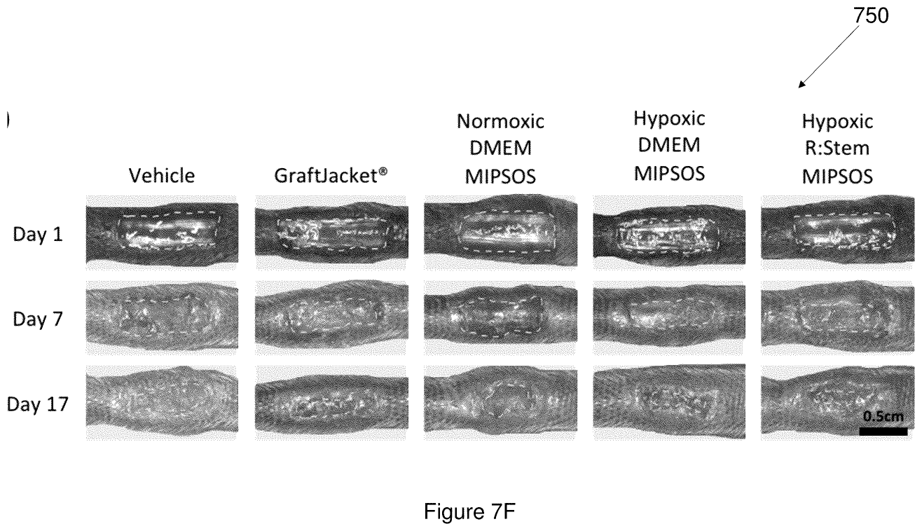

MIPSOS synthesised using DMEM/FBS, under normoxic or hypoxic conditions and MIPSOS synthesized in R: Stem under hypoxic conditions were investigated for their therapeutic efficacy in a full-thickness skin wounds in diabetic mice (FIG. 7A 700). Various MSC-derived matrices were mechanically scraped into deionised water and freeze-dried to obtain MIPSOS as typical insoluble microfragments. The dorsal side of the db/db mouse tail was chosen as a wound location, as it allows for sufficient surface area for critical size wounds, while the tail lacks the subcutaneous panniculus carnosus muscle layer and thus heals without contraction.

Two wounds, spaced 0.5 cm apart and with a surface area of 1 cm×0.3 cm, were created on the tail of mice (FIG. 7A 700). The different MIPSOS materials were delivered at a final concentration of 10 mg/ml in a fibrinogen solution (final concentration 10 mg/ml in 0.9% NaCl), polymerised by thrombin in situ. A clinically approved human dermal tissue-derived ECM (GraftJacket®), used in the treatment of chronic wounds, was chosen as a comparison. GraftJacket® was broken into microfragments and added at the same concentration into the wounds.

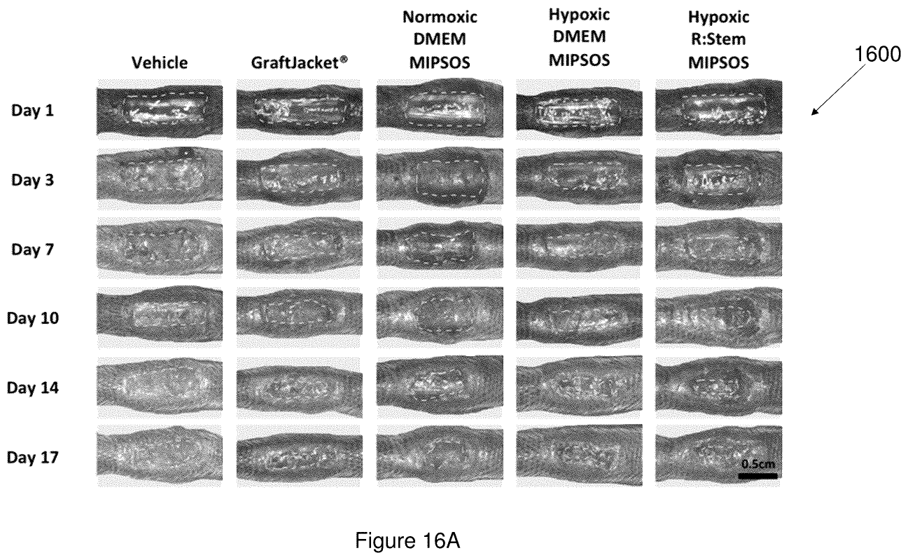

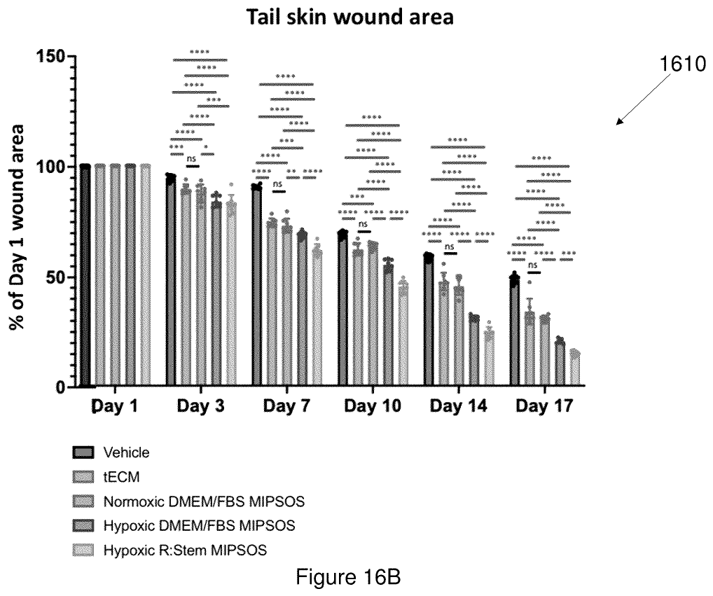

With reference to FIG. 7E 740 and FIG. 7F 750, untreated (vehicle only) wounds exhibited slow healing capability, resulting in a 50% wound closure after 17 days. Wounds treated with GraftJacket® and the original MIPSOS had comparable healing promoting effects and demonstrated an acceleration of wound closure from day 3 onwards, resulting in 65% wound closure by day 17 (FIG. 7B 710, 7C 720, FIG. 16A 1600). These effects were even more pronounced for MIPSOS assembled in R: Stem significantly outperforming MIPSOS assembled in DMEM/FBS from day 7 onwards (FIG. 7C 720, FIG. 16A 1600, FIG. 16B 1610). By day 17, wounds treated with MIPSOS synthesized under hypoxic conditions in DMEM/FBS or R: Stem closed by 80% and 85%, respectively (FIG. 7C 720). Simple linear regression analysis of the healing response revealed that the hypoxic R: Stem MIPSOS could significantly accelerate wound closure (FIG. 7D 730).

Congruently, immunohistochemical analysis of cytokeratin I (K1), a marker for mature epidermis, demonstrated full re-epithelialisation of wounds treated with MIPSOS synthesised under hypoxic conditions in DMEM/FBS and R: Stem. Comparable and partial re-epithelialisation (FIG. 8B2 811) was observed for wounds treated with MIPSOS produced under original conditions (normoxia & DMEM/FBS) and GraftJacket®.Semi-quantitative analysis of K1 positive tissue area is demonstrated corresponding trends (FIGS. 8A 800 and 8B1 810).

H&E staining of granulation tissues revealed the biologic composition as claimed synthesised under hypoxia, regardless of medium used, promoted cellular infiltration by day 17, outperforming the clinically approved GraftJacket® (FIG. 8C 820, 8D1 831, 8D2 830, 8D3 832). Masson Trichrome staining of granulation tissues showed increased collagen density in all treated wounds, as compared to the vehicle control (FIG. 8C 820, 8E1 841, 8E2 840, 8E3 842).

Semi-quantitative analysis of immunohistochemical staining of granulation tissue for CD31 revealed that the biologic composition significantly promoted higher vessel density, with wounds treated with MIPSOS synthesized under hypoxia exhibiting high endothelial cell density (FIG. 9A 900, 9B1 910, 9B2 911, 9B3 912, 9B4 913).

Advantageously, the increased cellular infiltration and revascularisation in wounds treated with MIPSOS synthesized under hypoxia were accompanied with an increased density of pro- and anti-inflammatory macrophages, as evident by the quantification of double positively stained iNOS/CD11b and CD206/CD11b cells in granulation tissue sections (FIG. 9A 900, 9B5 914, 9C1 920, 9C2 921, 9D1 940, 9D2 941). Surprisingly, although semi-quantitative analysis of F4/80+ cells showed a slight increase in cell numbers in the granulation tissues of wounds treated with GraftJacket® and MIPSOS synthesized with DMEM/FBS under normoxia as compared to that by vehicle only, the numbers were reduced when treated with corresponding materials synthesized under hypoxia. (FIG. 9B5 914, FIG. 9C1 920, FIG. 9C2 921, FIG. 9D1 940, FIG. 9D2 941, FIG.ue 9D3 940). A similar trend was observed for iNOS+ and CD206+ stained cells, indicating M1 and M2 macrophages, respectively.

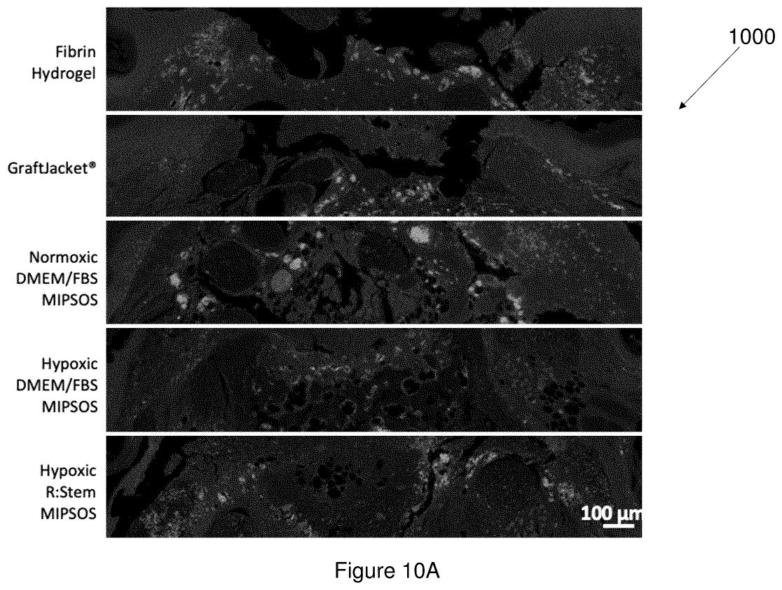

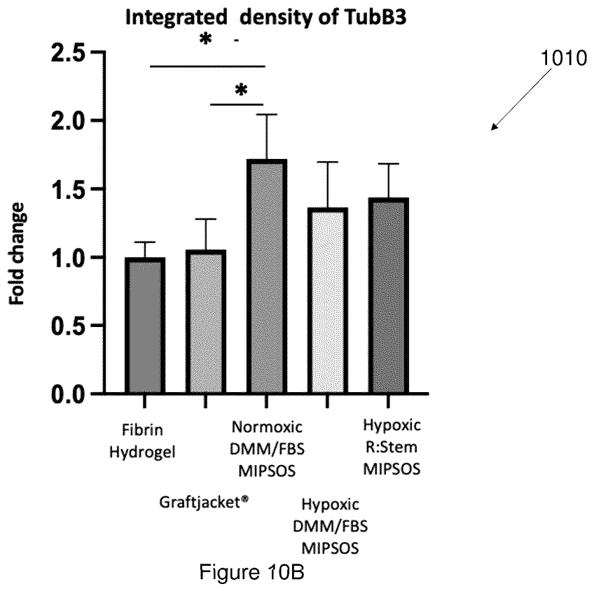

Immunohistochemical staining for β-tubulin III (TubB3), a neuronal microtubule protein, revealed the presence of regenerating neurites in the ingrowing epithelial tongues, as well as in the granulation tissue of healing wounds (FIG. 10A 1000). Enhanced reinnervation was observed in wounds treated with the biologic composition of the claimed invention (FIG. 10B 1010). This re-innervation promoting effect was not observed in wounds treated with GraftJacket® (FIGS. 10A 1000 and 10B 1010).

### DISCUSSION

Beneficially, the combination of hypoxia, a xeno-free, and/or serum-free, or chemically defined medium and the heparan sulfate mimetic, DxS, enabled the synthesis of the biologic composition of the claimed invention with augmented pro-angiogenic potential and pro-healing bioactivity, even exceeding the therapeutic efficacy of a clinically approved tissue-derived ECM biologic (GraftJacket®), an approved standard for the treatment of diabetic wounds.

The improved manufacturing protocol for MIPSOS enabled the synthesis of an MSC-derived ECM-based biologic of solely human origin, reducing the risk of adverse immunogenic responses and disease transmission, thereby paving the way for its GMP-compatible production and clinical translation. This careful selection of ancillary materials for the production of the biologic composition also provides increased safety and efficacy of the medicinal product.

All XS/SF media tested supported MSC proliferation and ECM deposition similarly or even better than DMEM/FBS, while all media compositions were compatible with DxS supplementation, which is crucial for the synthesis of an ECM-based biologic with augmented pro-angiogenic properties. Collagen I and fibronectin were chosen to assess the amounts of deposited ECM, as they, as structural components, comprise a large fraction of deposited ECM. The inventors found that different media had different effects.

Medium and culture conditions had a role to play in the gross composition of ECM, while MSCs cultured in R: Stem expressed more VEGF, a pan pro-angiogenic factor, which was further enhanced under hypoxia. VEGF accumulated in granular ECM structures, which were previously identified as aggregates of cell-derived ECM and DxS. DxS acts as a heparan sulfate mimetic and thus binds VEGF, while promoting accumulation of a wide plethora of pro-angiogenic factors in cell-derived ECM. It can be seen that culture conditions, which promote the synthesis of pro-angiogenic factors, act synergistically with DxS to facilitate their accumulation within cell-derived ECM.

The claimed invention thus provides a biologic composition with augmented pro-angiogenic properties in vitro and in vivo. In a diabetic wound healing model the improved MIPSOS preparation almost doubled the wound closure rate. For in vitro, the choice of culture medium had an effect on enhancing the bioactivity of MSC-derived ECM, while in vivo ECM synthesis under hypoxia most efficiently promoted cell infiltration, which led to enhanced re-vascularisation and re-epithelisation. Since cell migration and infiltration is severely impaired in diabetic wound healing, the increased density of pro- and anti-inflammatory macrophages pointed to a higher remodeling rate during wound healing and thus a more advanced healing progression. This was further evidenced by wounds treated with MIPSOS exhibiting a denser de novo deposited ECM and more enhanced skin re-innervation, both processes known to be normally impaired during diabetic wound healing.

The inventors have shown that the biologic composition (MIPSOS) promoted revascularization and healing in skin wounds of healthy and immunocompetent mice. The inventors further show that MIPSOS augmented diabetic wound healing in a murine model, which heals by cell migration instead of wound contraction. Although GraftJacket®, a current clinical standard, has previously been reported to promote satisfactory clinical outcome in chronic wound treatment, it is derived from decellularized human cadaveric dermis and poses limitations such as limited availability, risk of immunogenicity, batch-to-batch variability and the inability to tailor its bioactivity for desired properties. The biologic composition of the claimed invention advantageously accelerated wound closure by 40% as compared to GraftJacket®. Synthesis of MIPSOS in culture further enables a gentle and more controlled decellularisation, thereby reducing the risk of immunogenicity, while preserving bioactivity. Moreover, synthesis of the biologic composition allows for tailored bioactivity. The utilisation of MIPSOS for chronic wound treatment overcomes many of challenges faced by tissue-derived biologics, such as GraftJacket®. MIPSOS can retain its bioactivity for several weeks to months when stored frozen and desiccated, and is therefore a suitable composition with effective and widespread application. The biologic composition as claimed contains an enriched, complex portfolio of pro-reparative factors, whilst being of human origin and cell-free. Its utilisation can thus beneficially address major limitations of soluble factor-based, tissue-derived ECM-based therapies, as well as cell-based therapies. The successful production of MIPSOS, with further enhanced pro-angiogenic properties, under controlled and xeno-free conditions can thus pave the way for clinical translation for the treatment of non-healing chronic wounds.

1. A biologic composition for promoting wound healing, comprising:
a human mesenchymal stem cell (MSC)-derived extracellular matrix (ECM) biologic component, and
a negatively charged sulphated polymer,
wherein the negatively charged polymer is combined with the biologic component to form the biologic composition.

2. The biologic composition of claim 1, wherein the negatively charged polymer is a sulphated polymer.

3. The biologic composition of claim 1, wherein the negatively charged polymer is a polyglucose polymer.

4. The biologic composition of claim 1, wherein the negatively charged polymer is dextran sulfate (DxS).

5. The biologic composition of claim 1, wherein the biologic component is a secretome with pro-angiogenic factors.

6. The biologic composition of claim 1, wherein MSCs from which the biologic component is derived are cultured under xeno-free and/or serum-free conditions.

7. The biologic composition of claim 1, wherein MSCs from which the biologic component is derived are cultured in chemically defined conditions.

8. The biologic composition of claim 1, wherein MSCs from which the biologic component is derived are cultured in a chemically defined medium under hypoxic conditions.

9. The biologic composition of claim 1, wherein the biologic component is a secretome, and the secretome is aggregated and co-precipitated with the negatively charged polymer into pericellular space of hypoxia-primed MSC to form an insoluble ECM that is decellularized and processed into the biologic composition.

10. The biologic composition of claim 1, wherein the biologic composition is MicroParticles of Solidified Secretome (MIPSOS).

11. The biologic composition of claim 1, wherein the biologic composition is free of xeno- and serum-derived components.

12. The biologic composition of claim 1, wherein the biologic composition is obtained by one or more of: mechanical collection, solubilization, and lyophilization.

13. The biologic composition of claim 1, further comprising a carrier component to facilitate application and retention of the biologic composition at a target site.

14. The biologic composition of claim 13, wherein the carrier component is selected from hydrogels, sponges, sheets, powders, wound cleansers, antiseptic solutions, saline solution, topical antibiotics, barrier creams, emollients, hydrocolloid creams, hydrocolloid dressings, foam dressings, antimicrobial dressings, collagen dressings, dressings with a foam and film layer, and combinations thereof.

15. The biologic composition of claim 1, wherein the negatively charged polymer facilitates the deposition of an ECM with pro-angiogenic factors.

16. The biologic composition of claim 15, wherein the negatively charged polymer is 500 kDA dextran sulfate sodium salt from Leuconostoc spp which promotes the deposition and retention of bioactive factors within the ECM for augmented bioactivity.

17. The biologic composition of claim 1, further comprising one or more bioactive factors selected from: growth factors, cytokines, and chemokines.

18. The biologic composition of claim 1, wherein the biologic composition promotes angiogenesis in vitro and in vivo and accelerates re-epithelisation and wound closure.

19. The biologic composition of claim 1, wherein the biologic composition promotes one or more of the following in skin wounds: wound closure, revascularisation, cellular infiltration, ECM deposition, granulation tissue formation, and re-innervation.

20. A method for treating diabetic wounds, comprising administering to a wound an effective amount of the biologic composition of claim 1.

21. A method of preparing a biologic composition for promoting wound healing, comprising the steps of:
(i) isolating human mesenchymal stem cells (MSC) and subjecting the MSC to hypoxia-priming in a medium to stimulate the production of an MSC-derived biologic component with pro-angiogenic factors;
(ii) aggregating and co-precipitating the MSC-derived biologic component with a negatively charged polymer into a pericellular space of the MSC to produce an aggregated biologic component;
(iii) assembling the aggregated biologic component and the negatively charged polymer into an insoluble extracellular matrix (ECM) by the MSC; and
(iv) decellularizing the insoluble ECM to obtain the biologic composition comprising MicroParticles of Solidified Secretome (MIPSOS).

22. The method of claim 21, wherein the negatively charged polymer is a sulphated polymer.

23. The method of claim 21, wherein the negatively charged polymer is a sulphated polyglucose polymer.

24. The method of claim 21, wherein the negatively charged polymer is dextran sulfate (DxS).

25. The method of claim 21, wherein the medium is a xeno-free, and/or serum-free, or a chemically defined medium.

26. The method of claim 21, further comprising processing the insoluble ECM, by chemical or mechanical means, to obtain the biologic composition comprising MicroParticles of Solidified Secretome (MIPSOS).

27. The method of claim 21, further comprising incorporating a carrier means to facilitate application of the biologic composition at a wound site.

28. The method of claim 21, wherein the negatively charged polymer is 500 kDa dextran sulfate sodium salt from Leuconostoc spp.

29. The biologic composition of claim 1, wherein the MSCs are cultured in R: Stem medium under hypoxic conditions comprising 5% Oz.

30. The biologic composition of claim 1, wherein the biologic composition demonstrates superior wound healing efficacy with wound closure rates of up to 85% at 17 days.

31. The biologic composition of claim 1, wherein the biologic component comprises enhanced levels of vascular endothelial growth factor A (VEGF-A).

32. The biologic composition of claim 1, wherein the negatively charged polymer is present at a concentration of 10 μg/ml during ECM synthesis.

33. The biologic composition of claim 1, wherein the biologic composition retains bioactivity for several weeks to months when stored frozen and desiccated.

34. The method of claim 21, wherein the MSCs are cultured for a period of 6 days without media changes under hypoxic conditions comprising 5% O₂ tension.

35. The method of claim 21, wherein the medium is R: Stem medium, which is xeno-free, serum-free, and chemically defined.

36. The method of claim 21, wherein the medium further comprises ascorbic acid at a concentration of 30 μg/ml.

37. The method of claim 21, further comprising mechanically scraping the decellularized material, resuspending in deionised water, and lyophilising into fine powder microparticles.
